(19) United States
(12) Patent Application Publication
(10) Pub. No.: US 2002/0028457 A1
(43) Pub. Date: Mar. 7, 2002

(54) Single target counting assays using semiconductor nanocrystals

(75) Inventors: Stephen Alexander Empedocles, Mountain View, CA (US); Andrew R. Watson, Belmont, CA (US); Vince Phillips, Sunnyvale, CA (US); Edith Wong, Danville, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Quantum Dot Corporation, Hayward, CA (US)

(21) Appl. No.: 09/882,193

(22) Filed: Jun. 13, 2001

Related U.S. Application Data
(63) Continuation-in-part of application No. 09/784,866, filed on Feb. 15, 2001.
(60) Provisional application No. 60/182,844, filed on Feb. 16, 2000.
(60) Provisional application No. 60/211,054, filed on Jun. 13, 2000.

(51) Int. Cl. C12Q 1/68

(52) U.S. Cl. 435/6; 435/8

(57) ABSTRACT

The present invention provides assays that allow for the detection of a single copy of a target of interest. The target species is either directly or indirectly labeled with a semiconductor nanocrytal, “quantum dot.” The bright and tunable fluorescence of the quantum dot is readily detected using methods described herein. Also provided are assays that are based on the colocalization of two or more differently colored quantum dots on a single target species, which provides superbly sensitive assays in which the decrease in assay sensitivity caused by non-specific binding of assay mixture components to the assay substrate is minimized. The assays are of use to detect target species including, but are not limited to, nucleic acids, polypeptides, small organic bioactive agents (e.g., drugs, agents of war, herbicides, pesticides, etc.) and organisms.

### CROSS-REFERENCES TO RELATED APPLICATIONS

The application is a continuation-in-part of U.S. patent application Ser. No. 01/05164 filed on Feb. 16, 2001 which claims priority to U.S. Provisional patent application Ser. No. 60/182,844 filed on Feb. 16, 2000. The application also claims priority to U.S. Provisional Patent Application Ser. No. 60/211,054 filed on Jun. 13, 2000. The disclosures all of which are incorporated herein in their entirety for all purposes.

### BACKGROUND OF THE INVENTION

Bioassays are used to probe for the presence and/or the quantity of a target material in a biological sample. Surface-based assays, in which the amount of target is quantified by capturing it on a solid support and then labeling it with a detectable label, are especially important since they allow the easy separation of bound and unbound labels. One example of a surface-based assay is a DNA microarray. The use of DNA microarrays has become widely adopted in the study of gene expression and genotyping due to the ability to monitor large numbers of genes simultaneously (Schena et al., Science 270:467-470 (1995); Pollack et al., Nat. Genet. 23:41-46 (1999)). More than 100,000 different probe sequences can be bound to distinct spatial locations across the microarray surface, each spot corresponding to a single gene (Schena et al., Tibtech 16:301-306 (1998)). When a fluorescent-labeled DNA target sample is placed over the surface of the array, individual DNA strands hybridize to complementary strands within each array spot. The level of fluorescence detected quantifies the number of copies bound to the array surface and therefore the relative presence of each gene, while the location of each spot determines the gene identity. Using arrays, it is theoretically possible to simultaneously monitor the expression of all genes in the human genome. This is an extremely powerful technique, with applications spanning all areas of genetics (For some examples, see the Chipping Forecast supplement to Nature Genetics 21 (1999)). Arrays can also be fabricated using other binding moieties such as antibodies, proteins, haptens, aptazymes or aptamers, in order to facilitate a wide variety of bioassays in array format.

Other surface-based assays include microtitre plate-based ELISAs in which the bottom of each well is coated with a different antibody. A protein sample is then added to each well along with a fluorescent-labeled secondary antibody for each protein. Target proteins are captured on the surface of each well and secondarily labeled with a fluorophore. Fluorescence at the bottom of each well quantifies the amount of each target molecule in the sample. Similarly, antibodies or DNA can be bound to a microsphere such as a polymer bead and assayed as described above. Once again, each of these assay formats is amenable for use with a plurality of binding moieties as described for arrays.

Diagnostic assays that sensitively, specifically, and quickly detect pathogens in biological samples preferably use biopolymer receptors coupled with sensitive detection schemes. Few assays are able to detect physiologically or clinically relevant organic and protein concentrations on an appropriate time-scale for the early detection of the presence of an infective or otherwise harmful agent. To date, the most sensitive detection methods involve PCR, which is too complicated for use as a field assay and inherently misses non-nucleic acid signals associated with pathogenesis (e.g., bacterial toxins in the blood). Several reviews of progress in pathogen detection indicate that techniques like electrochemiluminescence (Yu et al, Biosensors and Bioelectronics 14:829 (2000)) (ECL) detect at best 10⁷ toxin molecules/ml, while potentiometric (Lee et al, Biosensors and Bioelectronics 14:795 (2000)) and photoluminescence (Koch et al., Biosensors and Bioelectronics, 14:779 (2000)) detection yields 10⁹ and 10¹⁰ molecules/ml respectively. A broad review of affinity-based biosensors suggests that even the most sensitive methods (e.g., amperometric immunosensors) detect only 10⁶ olecules/ml (see, Rogers, Mol. Biotechnol. 14: 109 (2000)). In other words, the routine detection of hundreds to thousands of biopolymer:analyte interactions in a milliliter of sample is still extremely difficult.

The most important characteristics of a bioassay are sensitivity, specificity and dynamic range. The performance of an assay is typically measured by its ability to specifically and quantitatively measure vanishingly small quantities of assay material. This is especially true for genetic analysis such as gene expression or genotyping, where the available quantity of genetic material is limited. For instance, using current detection technology with organic dye labels, gene expression analysis on DNA microarrays requires between 50 and 200 μg of total RNA for a single array hybridization. This requires as many as 10⁵ cells (Duggan et al., Nature Genetics 21(n1 s):10-14 (1999)). In many cases, such as samples extracted through microdissection (Sgroi et al., Cancer Res. 59:5656-5661 (1999)), these large quantities of material are not available. This greatly complicates the detection of such samples labeled with standard organic fluorophores.

The primary shortcoming of surface-based assays such as DNA microarrays is the lack of appropriate sensitivity needed to detect extremely low levels of target concentration. For instance, as much as 40% of the known genes of interest studied using gene expression microarrays are expressed at a level of between 1 and 10 copies per cell, just at or below the limit of detection using current detection schemes. In addition to low expression levels, the costs incurred in extracting material for genetic testing is creating pressure to minimize sample size requirements for genetic analysis. Currently, the preferred method for detection of surface-based assays such as microarrays is by labeling target molecules with organic dyes. For DNA microarrays using organic dyes, the current state-of-the-art detection can only detect a minimum of approximately 10 molecules in a 10 μm×10 μm region of a microarray spot (Duggan et al., Nature Genetics 21(n1s): 10-14 (1999)). Thus, the minimum number of bound DNA molecules required in order to detect signal from a standard 100μm diameter microarray spot is approximately 1000. In order to generate a signal of detectable intensity, more than 10 million cells may be required. In many instances, it is not possible to extract this much cellular material. Thus, methods for enhancing the sensitivity of assay detection are needed.

Dynamic range refers to the ability to simultaneously measure analyte over a wide range of concentrations. Using current detection technology, it is usually necessary to sacrifice linearity in the high concentration regime for detection sensitivity in the low concentration regime. This limits the dynamic range of a single experiment.

In order to improve existing surface-based bioassays, it is necessary to improve both sensitivity and dynamic range. The invention disclosed here describes a method for detecting and counting single bound target molecules in surface-based assays. This will dramatically increase both the sensitivity and dynamic range of these bioassays.

In many instances, the sensitivity of a bioassay is not limited by the ability to detect the assay signal, but by interference from nonspecifically bound target molecules and/or labels. The fundamental limit of assay sensitivity under a certain set of assay conditions is defined by the concentration at which a decrease in concentration results in a change in signal that is undetectable above the noise generated by nonspecifically bound labels. This limit is independent of the method of label detection and may occur at a concentration that is either higher or lower than the limit of label detection. Using traditional detection techniques, it is not possible to detect beyond the non-specific binding limit. The current invention provides a method by which this limit can be passed and even eliminated, dramatically improving detection sensitivity in a variety of surface-based assays.

### BRIEF SUMMARY OF THE INVENTION

The present invention provides methods of increasing the sensitivity, specificity and dynamic range of assay detection. The methods of the present invention allow for the detection of individual copies of a target species or target nucleic acid sequence present in an assay mixture (“single target counting”). In a surface based assay, using single target counting, the theoretical limit of detection is 1 molecule in the binding region, dramatically reducing the amount of target species required for detection relative to ensemble detection techniques. The ability to detect single target molecules in all types of assays dramatically improves the sensitivity and dynamic range of the assays, thereby enhancing the information content and the minimizing cost of the assay.

Thus, in a first aspect, the present invention provides a method of detecting a single copy of a target species. The method includes detecting a single copy of the target species by detecting fluorescence emitted by a quantum dot attached, either directly or indirectly, to the single copy. The single copy is bound to an affinity moiety for the target species, which recognizes and selectively interacts with the target species.

In a second aspect, the invention provides a method of detecting a first target species immobilized on a substrate. The method includes: (a) defining a first region of interest of the substrate; and (b) probing the first region of interest for fluorescence emitted by a quantum dot attached, either directly or indirectly, to a single copy of the target species bound to an affinity moiety for the target species, which is immobilized on said substrate.

In a third aspect, the present invention provides a method for quantifying a target species immobilized on a substrate. The method includes: (a) detecting fluorescence emitted by a quantum dot attached, either directly or indirectly, to a single copy of the target species bound to an affinity moiety for the target species immobilized on the substrate; (b) counting each detected quantum dot per unit area of the substrate, producing substrate quantum dot data; and (c) comparing the substrate quantum dot data with standard quantum dot quantity data acquired from a standard of the quantum dot-labeled target having a known concentration of target molecules, thereby quantifying the target species immobilized on said substrate.

In a fourth aspect, the invention provides a method of detecting a target species immobilized on a substrate, which is a member of a population of target species immobilized on the substrate with spacing between each member of the population. The method includes, detecting a single copy of the target species by detecting fluorescence emitted by a quantum dot attached, either directly or indirectly, to the single copy. The single copy is bound to an affinity moiety for the target species, which is immobilized on the substrate. The detecting is performed with a detecting means having a resolution that is higher than the spacing between each member of the population, such that the signal from each bound target molecule can be substantially detected and distinguished from the surrounding bound target molecules.

In a fifth aspect, there is provided a method of detecting a target species immobilized on a substrate, which is a member of a population of target species immobilized on the substrate. The method includes, detecting a single copy of the target species by detecting fluorescence emitted by a quantum dot attached, either directly or indirectly, to the single copy. The single copy is bound to an affinity moiety for the target species, which is immobilized on the substrate, thereby forming a target-affinity moiety complex. The detecting is performed with a detecting means having a resolution limited region of interest such that less than one target-affinity moiety complex is present within each resolution limited region of interest.

In a sixth aspect, the invention provides a method of detecting a first target species immobilized on a substrate, which is a member of a population of target species immobilized on said substrate. The method includes: (a) defining a first region of interest of the substrate; (b) probing the first region of interest for fluorescence emitted by a quantum dot attached, either directly or indirectly, to a single copy of the target species bound to an affinity moiety for the target species immobilized on the substrate. The probing resolves the fluorescence from the target species from fluorescence arising from other members of the population of target species immobilized on said substrate.

In an seventh aspect, the invention provides a method for detecting multiple target species immobilized on a substrate, which are members of a population of target species immobilized on said substrate. The method includes: (a) defining multiple regions of interest on the substrate; and (b) probing the multiple regions of interest for fluorescence emitted by a quantum dot attached, either directly or indirectly, to a single copy of the target species bound to an affinity moiety for the target species immobilized within a region of interest of the substrate. The probing resolves fluorescence from the multiple target species from other members of the population.

In a eighth aspect, the invention provides a method for determining whether a target species within a region of interest on a substrate is quantifiable by a technique selected from the group consisting of single target counting and ensemble intensity detection. The method includes: (a) probing the region of interest to determine target species density within the region of interest by detecting fluorescence emitted by a quantum dot attached, either directly or indirectly, to one or more molecules of the target species bound to an affinity moiety for the target species immobilized on the substrate; (b) comparing the density to a predetermined density cutoff value above which ensemble intensity detection is used and below which single target counting is used.

In a ninth aspect, the invention provides a method for differentiating specific binding of target species to the assay substrate from nonspecifically bound target molecules and from nonspecifically bound label species. The method includes: (a) binding said target species to an affinity moiety attached to a substrate, said target species independently labeled with two or more quantum dots with distinguishable fluorescence, (b) identifying single target species by the simultaneous presence of both quantum dot signals associated with each target species.

In a tenth aspect, the invention provides a method of detecting a target species in solution. The method includes, detecting a single copy of the target species by detecting essentially simultaneously fluorescence emitted by a first quantum dot of a first color attached, either directly or indirectly, to the single copy and a second quantum dot of a second color attached, either directly or indirectly, to the single copy, wherein the first color and the second color are distinguishably different colors.

Other objects and advantages of the present invention will be apparent from the detailed description that follows.

### BRIEF DESCRIPTION OF THE DRAWINGS

FIG. 1 Single quantum dot detection. (A) Image of single quantum dots using a laser epifluorescence microscope. Each individual spot corresponds to the fluorescence from a single quantum dot. (B) Spectra from single quantum dots. Wavelength is dispersed on the x-axis and position on the y-axis. Each horizontal line corresponds to the fluorescence spectrum from a single quantum dot. Note that different size quantum dots are easily identified by small changes in emission wavelength.

FIG. 2 Dynamic range of ensemble intensity detection and single target counting. (A) Graphic representation of the transition from the ensemble concentration regime to the single target counting regime. (B) Simulated data demonstrating the improved sensitivity reached through single target detection. (C) Theoretical number of discrete points detected within a 100 μm diameter spot as the density of bound labels increases.

FIG. 3 Preliminary single target counting assay. (A) Images of assay substrates that were washed with different concentrations of target. Individual spots within each image correspond to single target molecules. (B) Titration curve for the data displayed in (A).

FIG. 4 Receptor binding to (A) individual epitopes of a molecular target; and (B) to multiple, identical surface proteins on a cellular target.

FIG. 5 is a schematic diagram of an exemplary quantum dot detection apparatus.

FIG. 6 Single target coincidence staining. Top spectra indicate the fluorescence detected with a high resolution imaging system. Each target and label is resolved and specific signal is identified by 2 colors. The bottom spectrum indicates the average spectrum from the entire image as detected with a low-resolution imaging system. Both specific and non-specific signal contribute to the bottom spectrum, blurring the distinction between specific and non-specific signal.

FIG. 7 SAC² (“single analyte coincidence staining and counting”) detection and analysis by eye. By using combinations of colors to label each specific target, it is possible to perform single-analyte coincidence measurements by eye, facilitating a manual, portable detection system.

FIG. 8 Automated array scanning. (A) sequential images are taken at periodic positions across the array. (B) The array image is reconstructed. (C) Pattern recognition identifies location of array spots relative to “alignment spots.” (D) Within each spot the average intensity is measured as well as the total number of discrete points. (E) Both values are exported.

FIG. 9 Identification of specific assay signal in the presence of non-specific signal using SAC². Three molecules are bound to the assay surface by binding receptors: two “specific” targets and one non-specifically bound target. There is also a non-specifically bound label. Both specific targets are identified by the presence of 2 colors (i.e. a coincidence signal), while the non-specific signals have only one. Spectra represent the detected emission spectra for each signal.

FIG. 10 An exemplary data extraction and analysis procedure of use with the present invention.

FIG. 11 Simple assay processing.

FIG. 12 Transcription of the K-ras DNA target, having 3 point mutations, by the biotinylated primer.

FIG. 13 Transcribed, biotinylated target captured onto a solid support coated with streptavidin.

FIG. 14 Captured sample strands aligned on the surface.

FIG. 15 Probing of the captured strands by sequence-tagged hybridization probes.

FIG. 16 Six colors of quantum dots bound to the capture sequence tags.

FIG. 17 An image from scanning with sufficient resolution to detect single quantum dots. The mutant target is identified via the binding and subsequent co-localization of multiple oligo probes, each containing a different color reporter quantum dot. The combination of co-localized colors bound indicates which mutations are present.

### DETAILED DESCRIPTION

Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization described below are those well known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications such as capping with a fluorophore (e.g., quantum dot) or another moiety.

“Peptide” refers to a polymer in which the monomers are amino acids and are joined together through amide bonds, alternatively referred to as a “polypeptide.” Unnatural amino acids, for example, β-alanine, phenylglycine and homoarginine are also included under this definition. Amino acids that are not gene-encoded may also be used in the present invention. Furthermore, amino acids that have been modified to include reactive groups may also be used in the invention. All of the amino acids used in the present invention may be either the D-or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

“Antibody,” as used herein, generally refers to a polypeptide comprising a framework region from an immunoglobulin or fragments or immunoconjugates thereof that specifically binds and recognizes an antigen. The recognized immunoglobulins include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

As used herein, “fragment” is defined as at least a portion of the variable region of the immunoglobulin molecule, which binds to its target, i.e. the antigen binding region. Some of the constant region of the immunoglobulin may be included.

As used herein, an “immunoconjugate” means any molecule or ligand such as an antibody or growth factor (i.e., hormone) chemically or biologically linked to a fluorophore, a cytotoxin, an anti-tumor drug, a therapeutic agent or the like. Examples of immunoconjugates include immunotoxins and antibody conjugates.

The term “alkylene” by itself or as part of another substituent means a divalent radical derived from an alkane, as exemplified by—CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) 0, N and S may be placed at any interior position of the heteroalkyl group. The heteroatom Si may be placed at any position of the heteroalkyl group, including the position at which the alkyl group is attached to the remainder of the molecule. Examples include—CH₂—CH₂—O—CH₃, —CH₂—CH₂—NH—CH₃, —CH₂—CH₂—N(CH₃)—CH₃, —CH₂—S—CH₂—CH₃, —CH₂—S(O)—CH₃, —CH₂—CH₂—S (O)₂—CH₃, —CH=CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified by —CH₂—CH₂—S—CH₂CH₂—and —CH₂—S—CH₂—CH ₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied.

Each of the above terms are meant to include both substituted and unsubstituted forms of the indicated radical.

As used herein, the term “heteroatom” is meant to include oxygen (O), nitrogen (N), sulfur (S) and silicon (Si).

The term “affinity moiety” refers to a species, which is a functional group, a molecule, a cell, an organism or a combination of these species. The “affinity moiety” recognizes a target in an assay mixture and binds or otherwise interacts with the target. The interaction between the target and the affinity moiety is an event made detectable by the presence of a fluorophore (e.g., quantum dot) attached, either directly or indirectly, to one or more of the affinity moiety, the target, or an intermediate ligand that interacts with either or both the affinity moiety and target. An affinity moiety can be bound to, or otherwise associated with, a substrate, or it can be free in solution.

“Target,” and “target species”, as utilized herein refers to the species of interest in an assay mixture. Exemplary targets include, but are not limited to cells and portions thereof, proteins, nucleic acids, DNA, RNA enzymes, antibodies and other biomolecules, drugs, pesticides, herbicides, toxins, small molecules, agents of war and other bioactive agents.

The term, “assay mixture,” refers to a mixture that includes the target and other components. The other components are, for example, diluents, buffers, detergents, and contaminating species, debris and the like that are found mixed with the target. The other components may also include a biological matrix such as blood, plasma, semen, homogenized tissue or other biological fluid.

As used herein, “reactive spacer” refers to species that have a functional group available for reaction with an affinity moiety.

“Epitope,” as used herein refers to a characteristic, on either molecules or cells, recognized by a binding-receptor (e.g., an affinity moiety).

The term “ion pair” is meant to include salts formed between the target and the affinity moiety. When the affinity moiety or target contain relatively acidic functionalities, base addition salts can be obtained by contacting the neutral form of such compounds with a base. Examples of counter-ions in salts of acids include, sodium, potassium, calcium, ammonium, organic amino, magnesium, or a similar salt. When either the affinity moiety or the target contain relatively basic functionalities, acid addition salts can be obtained by contacting the neutral form of such compounds with an acid. Examples of counter-ions in salts of bases include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al, “Pharmaceutical Salts”, Journal of Pharmaceutical Science 66: 1-19 (1977). Certain affinity moieties or targets may contain both basic and acidic functionalities that allow the compounds to be converted into either base or acid addition salts.

The term “drug” or “pharmaceutical agent,” refers to bioactive compounds that cause an effect in a biological organism. Drugs used as affinity moieties or targets can be neutral or in their salt forms. Moreover, the compounds can be used in the present method in a prodrug form. Prodrugs are those compounds that readily undergo chemical changes under physiological conditions to provide the compounds of interest in the present invention.

“Organism,” as used herein, refers to viruses, bacteria, fungi, single-and multi-cellular life forms and cells derived from multi-cellular life forms.

The terms “ensemble regime,” and “ensemble counting,” are used interchangeably herein and refer to detection of signal from a plurality of detectably labeled targets in the field, e.g., an array spot, typically relatively homogenously dispersed within the field, in the form of average emission intensity over the area of the detection field. In his regime, sample concentration is proportional to average emission intensity.

The term “standard quantum dot quantity data,” refers to concentration data that is acquired using any of the methods described herein using a solution of target molecules in which the concentration of at least one target molecule is known or a substrate that has immobilized thereon target molecules from a solution of target molecules in which the concentration of at least one target molecule is known.

Introduction

The invention disclosed herein includes methods for increasing the sensitivity, specificity and dynamic range of assay systems based upon the capture of a target species with an affinity moiety. The assays can be surface based, in which a component of the assay (e.g., an affinity moiety) is bound to a substrate. Alternatively, the interaction between the affinity moiety and the target species can occur in solution.

The present invention is further explained and illustrated by reference to a preferred embodiment in which the methods of the invention are practiced in a fluorescent surface-based assay using a quantum dot as the fluorophore. This focus is for purposes of clarity and simplicity of illustration only, and should not be construed as limiting the scope of the present invention or circumscribing the types of assays in which the present invention finds application. Those of skill in the art will recognize that the methods set forth herein are broadly applicable to a number of assay systems, using any fluorophore detectable at the single molecule level, and in the detection of a wide range of target moieties.

Moreover, the methods and assays described herein do not actually require the ability to detect a single label (e.g. a single quantum dot). The invention is preferably practiced by detecting a single target species (e.g., molecule, cell, etc.). Therefore, the methods described herein are used to detect single target species that are labeled with a single detectable label, or with multiple detectable labels. Thus, one of skill in the art will appreciate that those methods of the invention described by focusing on species labeled with a single fluorophore can also be practiced with species labeled with two or more fluorophores.

A. Quantum Dots

The single target counting method and assays utilizing this method described herein can be performed using any fluorescent label capable of being detected on the single molecule level. Exemplary fluorophores include, but are not limited to organic dye molecules, metal colloid scattering particles, and surface-enhanced Raman spectroscopy (SERS) particles. Semiconductor nanocrystal labels (“quantum dots”) are a presently preferred fluorophore for use in the invention. As described below, semiconductor nanocrystals have many extraordinary optical characteristics that make them ideal for use as labels in the present single target counting methods and in assays applying these methods.

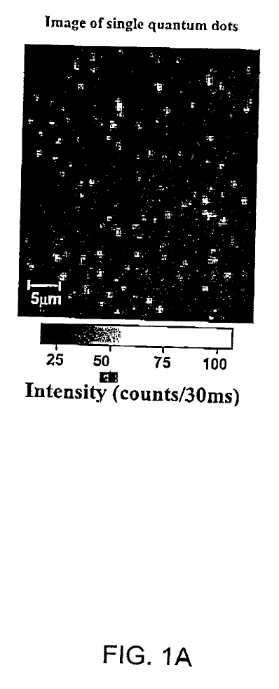

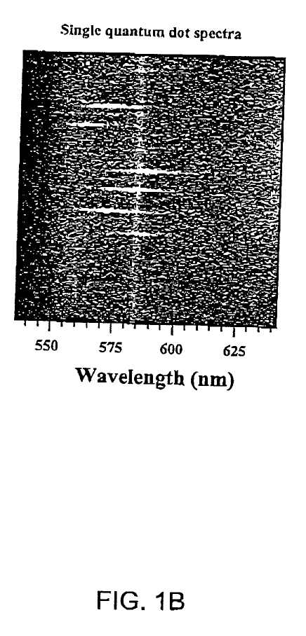

Quantum dots are a presently preferred fluorophore for use in the methods of the invention. Fluorescence from semiconductor nanocrystals is extremely bright and stable, allowing the routine detection of the fluorescence from single semiconductor nanocrystals (FIG. 1). Moreover, because the fluorescence of quantum dots can be “tuned” over a broad emission wavelength range, quantum dots are useful in multiplexing assays in which it is desired to detect more than one species based on differences in the fluorescence emission of the fluorophores bound to the species or alternatively detecting a single species using more than one fluorophores per species. Furthermore, emission wavelengths can be selected to avoid overlap with autofluorescence. In addition, since semiconductor nanocrystals can also be excited at any wavelength shorter than the emission wavelength, excitation can also be chosen to avoid exciting autofluorescence. Appropriately chosen excitation and emission wavelengths can dramatically reduce autofluorescence, increasing detection sensitivity. See, generally, Empedocles et al., Nature 399: 126-130 (1999); Empedocles et al, Acc. Chem. Res. 32: 389-396 (1999); Empedocles et al., Science 278: 2114-2117 (1997); Empedocles et al., Phys. Rev. Lett. 77: 3873-3876 (1996); Alivisatos, Science 271: 933-937 (1996); Efros et al., Sov. Phys. Semicond. 16:772-775 (1982); Hines et al., J. Phys. Chem. 100: 468-471 (1996); Peng et al., J. Am. Chem. Soc. 119: 7019-7029 (1997); Dabbousi et al., J. Phys. Chem. B 101: 9463-9475 (1997); Bruchez et. al, Science 281: 2013-2016 (1998); and Chan et. al., Science 281: 2016 (1998).

High stability, detection sensitivity and ease of multiplexing make semiconductor nanocrystals the preferred multi-color fluorophores for use in ultra-sensitive assays (e.g., surface-based assays). The ability to easily detect single semiconductor nanocrystals makes quantum dots a preferred fluorophore for use in assays using single target detection (e.g., bioassays) in which single target molecules bound to an affinity moiety are counted one at a time.

B. Single Target Detection “Single target counting,” or “single target detection,” as used herein refers to the counting of individual copies of a target species. In a preferred embodiment, the target species interact with an affinity moiety that is immobilized on a substrate, Following their being anchored to the substrate via the affinity moiety, the individual target species are detected by detecting the fluorescence or the change in fluorescence of a fluorophore. The fluorophore is preferably attached, either directly or indirectly, to the affinity moiety, the target or a combination thereof. In another embodiment, a fluorophore is attached to a third group that interacts with the target, the affinity moiety, the target-affinity moiety complex (e.g., sandwich assay), or combinations thereof.

In the surface-based embodiment of the invention, the species that are counted individually are generally those anchored to the surface via their interaction with the surface-bound affinity moiety. The method of the invention does not require the individual counting of all the target species within a sample. While the number of targets immobilized onto a substrate and the number of targets in the sample is typically not the same, as with any assay, the actual target concentration in the sample solution can be determined through calibration against a sample of known concentration. By enabling the detection and counting of single bound target molecules, the present invention extends the sensitivity of assays beyond what is presently possible using current detection techniques.

By way of illustration, the sensitivity of surface-based assays such as microarrays can be extended by the use of single target counting. For instance, current microarray technology allows the detection of target at a density of as low as 0.1 labels/μm² (˜8 labels per 10 μm diameter confocal spot). In contrast, with single target counting, the theoretical limit of detection is 1 label per array spot, extending the detection sensitivity by as much as 3 orders of magnitude for a 100 μm diameter array element. It is within the scope of the present invention to utilize single target detection to improve the sensitivity of microarray-based assays as well as other assay formats known in the art. The use of the present invention in exemplary microarrays is described in commonly owned U.S. Provisional Pat. App. Ser. No. 60/182,845 filed on Feb. 16, 2000.

In order to understand how the detection of single bound target molecules improves the sensitivity and dynamic range of a surface-based assay, it is important to understand what is actually measured at the high and low end of the concentration range on an assay surface. Single target detection is illustrated by way of an exemplary surface-based microarray assay applying the single target counting method of the invention, however, the underlying conceptual framework is equally applicable to any assay format.

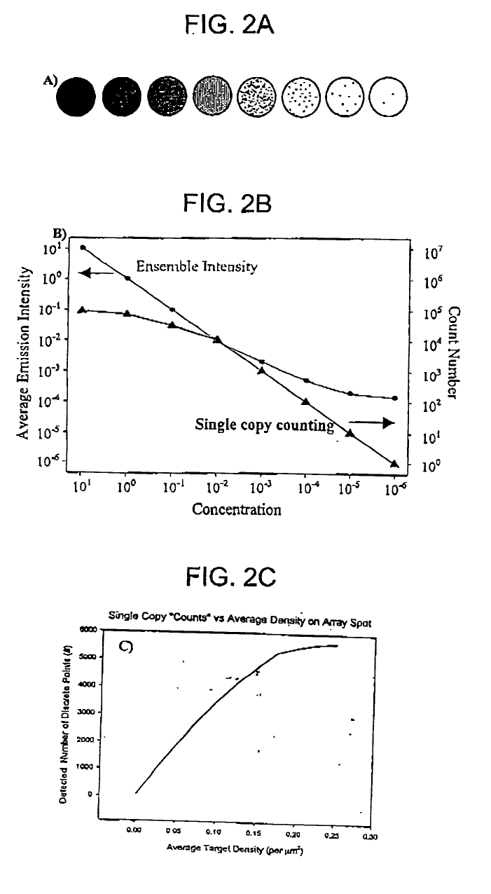

FIG. 2A is a graphic representation of a series of microarray spots with decreasing concentrations of bound target. The bound target on the left side is in the high concentration regime (“ensemble regime”) where the entire array spot is covered with target and the average emission intensity is dependent on the average density of label across the surface of the array. In this regime, sample concentration is proportional to average emission intensity (“ensemble intensity”). On the right side, the bound target is in the single target counting regime, where individual bound target molecules are separated from each other by distances that are greater than the resolution limit of the detection system and can be detected one at a time. In this regime, sample concentration is proportional to the number of individual targets counted on the surface of the array.

FIG. 2B shows data simulating the relative signal vs. concentration detected using ensemble intensity and single copy counting over the entire concentration range. Ensemble measurements yield linear concentration dependence at high concentrations, but saturate at low concentrations. This saturation occurs when the total signal from bound target in the detection region is lower than the noise generated from the integrated background across that entire region. Detecting single molecules bound to the array with high-resolution microscopy, however, can dramatically reduce the integrated background noise by comparing the signal from a single fluorophore to the background from an extremely small (potentially diffraction limited) area of the array spot.

As an example, if the background signal increases linearly with total detection area, then the background generated over a standard 10 μm diameter ensemble probe spot is 400 times higher than the background generated from a high resolution image of a single fluorophore (˜0.5 μm diameter). This results in a decrease in noise (and therefore an increase in sensitivity) of a factor of 20. This effect is further enhanced if the ensemble signal is integrated over the entire array spot. For a 100 μm diameter spot, the background signal is 40000 times higher than for a diffraction limited spot resulting in approximately 200 times higher sensitivity. The background over the bottom of an entire well of a 96 well plate is ˜10⁸ times higher yielding an enhancement of 10⁴. To achieve these enhancements, however, it must be possible to detect the fluorescence from a single bound target molecule with high spatial resolution.

In contrast to ensemble intensity measurements, the single target counting signal saturates at high concentrations. This occurs when the concentration increases to the point where individual target molecules are so close together that they cannot be distinguished. This means that some individual spots actually contain more than one bound target molecule and, therefore, counting the number of discrete points per unit area results in an undercounting of the total number of bound target molecules. The result is an underestimate of the total sample concentration (FIG. 2C).

Between the ensemble and single target counting regime, there is a regime in which the concentration is low enough to count individual targets, but high enough to be detectable in an ensemble measurement. This is referred to as the “transition regime.” The transition regime can be calibrated using ensemble and/or single target counting, allowing the user to calibrate concentrations across all regimes.

By combining single copy counting and ensemble intensity measurements, it is not only possible to increase detection sensitivity, but also the dynamic range of surface-based assays. In standard measurements, detection sensitivity at the low end is achieved at the expense of dynamic range at the high end due to detector saturation. The combination of single target counting with ensemble intensity measurements, however, can cover the entire dynamic range in a single experiment. In the single copy counting regime, as the concentration increases, the peak intensity does not; only the number of detected spots increases. As such, the entire dynamic range of the detector can be used to cover the ensemble concentration regime, where peak intensity varies linearly with concentration.

The embodiments discussed above focus on quantum dot-labeled targets. Other assay formats in which other assay components in addition to, or instead of, the target (e.g., affinity moiety) are labeled with a quantum dot are encompassed within the invention.

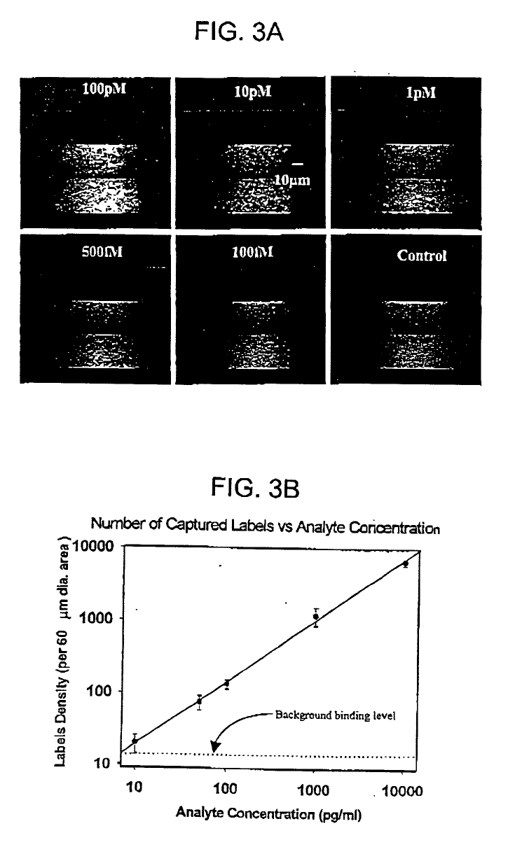

In an exemplary assay using single target counting of molecular or cellular targets, a dense layer of polyclonal anti-rabbit IgG was passively adsorbed to the surface of standard glass coverslips. Excess antibody was removed and the surfaces were blocked with BSA. Each coverslip was immersed in different concentrations of biotinylated rabbit IgG (10 nM to 100 fM plus PBS control). After binding for 15 minutes, the samples were washed and labeled with streptavidin functionalized quantum dots. After 30 minutes of washing in PBS/1%BSA/0.1% Igepalo at room temperature, samples were imaged with a fluorescence microscope. The points of light in FIG. 3A are signal from single bound analyte molecules, and the density of molecules can be seen decreasing as a function of analyte concentration. The assay was quantified by counting analyte molecules in a defined area. FIG. 3B shows the linearity and sensitivity of this simple assay to densities below 0.001 molecules/μm².

Detection of the single targets of an assay is accomplished by any method appropriate to the particular assay. Specific methods of detection are discussed in detail in Section D, infra.

In an exemplary embodiment, single target species labeled with quantum dots are easily detected by eye with the aid of a simple optical microscope, requiring no electronics. The concept and application of detection ‘by eye’ is illustrated by an exemplary assay of the invention, which is formatted as an “early-warning system,” providing a warning of exposure to a harmful agent such as a pesticide, herbicide, industrial pollutant, agent of war or pathogen, etc. In such a system, only a yes/no answer to whether there has been exposure to the harmful agent is required. The answer is easily supplied by comparing the density of spots in an assay to a threshold value. In this embodiment of the invention, the structure upon which the assay is performed can be incorporated into a number of devices including, but not limited to, wearable badges, hand-held detectors, and devices mounted to a wall, vehicle interior and the like.

C. Single Analyte Coincidence Staining and Counting (“SAC²”)

In addition to methods in which a single quantum dot of one color is used to label a component (e.g., target species) of an assay, the invention also provides methods in which two or more quantum dots of different colors are used to label a component. The use of more than one color of quantum dot per target provides assays in which specificity is dramatically increased, by requiring that the different colors or color combinations of the quantum dots coincide spatially during detection. This can dramatically reduce or even eliminate the detection of nonspecifically bound targets or labels, enhancing specificity and sensitivity of the assay. Underlying the improvement represented by SAC² is the improbability of accidentally encountering two or more preselected different colors at the same location at the same time. The improbability increases as more quantum dots of different colors are used. Alternatively, in another exemplary embodiment, the emission from the two or more differently colored quantum dots combines to form a third color, which is not otherwise present in the assay.

As discussed above in the context of single target detection, SAC² can be applied to substantially any assay of any format. For purposes of illustration, assays using SAC² are exemplified herein by the detection of pathogens and bioactive small molecules, such as might be used in warfare or terrorist attacks. The focus of the discussion that follows is for clarity of illustration and is not intended to define or limit the scope of the present invention or the scope of the targets that the present invention is useful to detect.

By detecting the fluorescence from individual labels and counting analyte molecules and organisms captured on an assay substrate, detection sensitivity of the present assays can be enhanced by about 2-3 orders of magnitude OR MORE over traditional detection techniques. Using the methods of the present invention, individual proteins have been detected at a surface binding-density of about 100-times lower than is detectable with traditional techniques (FIG. 3). The present invention provides methods to detect molecules such as toxins, and organisms such as bacteria, at concentrations in the body, which are preferably below 1000- and 100-per milliliter, respectively, extremely relevant concentrations for the early detection of infection.

In an exemplary application of the SAC² method, different features of a cell or epitopes of a molecule are labeled with different quantum dot colors. The target is detected and its identity is confirmed using the colocalization or “coincidence” of each color on each target. Coincidence staining allows for the detection and differentiation of different organisms or strains of organisms expressing different surface markers. Moreover, coincidence staining provides a method of distinguishing molecules of different structure down to the level of isomeric differences and differences in stereochemistry. The combination of coincidence staining with such single target counting provides an extremely powerful assay system.

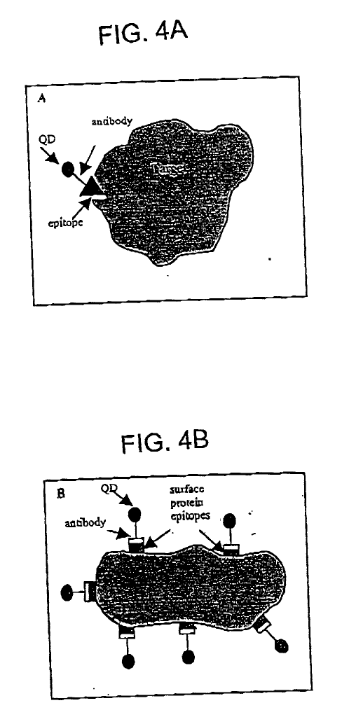

In the detection of pathogenesis, the most direct analyte is the pathogenic organism itself. In this case, assays preferably identify particular features of the organism such as surface proteins. To further aid in characterization, it is preferred to assay for molecular analytes as well. An example of a molecular analyte is an exotoxin such as cholera toxin. Antigen specific binding receptors are generated that recognize different characteristics of an analyte with high specificity. In the case of molecular analytes, receptors recognize different epitopes of a protein or small molecule (FIG. 4A), while cellular analytes are recognized through different molecules on the cell surface (FIG. 4B).

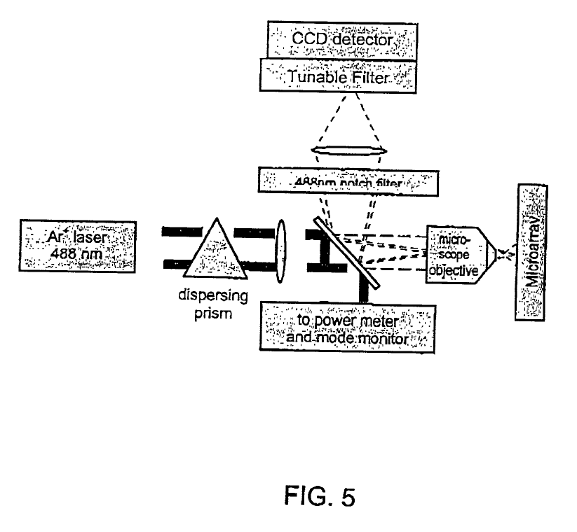

To facilitate coincidence staining, it is preferred to detect the fluorescence from each analyte independently. Thus, individual molecules or cells are preferably captured at a density that is low enough so that they are spatially resolved by the detection system (FIG. 5). This is well suited for use in combination with single analyte counting.

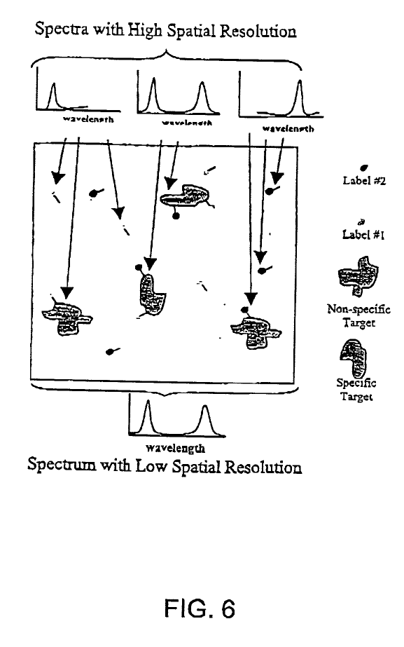

Single analyte coincidence staining can provide an assay that is even more sensitive than single target counting. In another exemplary embodiment, SAC² is used to differentiate between the formation of a target-affinity moiety complex and non-specific binding of the target to another component of the assay system. The intrinsic sensitivity of an assay often is limited by non-specific binding of the target or other assay mixture components to the substrate. Single analyte coincidence staining can be used to differentiate between specific binding of the target to the affinity moiety and non-specific binding of assay mixture components to the substrate based on the colocalization of quantum dot colors (FIG. 6). Those of skill in the art will appreciate that coincidence staining as described herein is useful to distinguish non-specific binding in solution-based assays as well.

SAC² can also be used to identify a single target. For example, one may wish to confirm the presence of a selected target in a mixture of targets that are structurally similar (e.g. having a common epitope) or that have similar affinity for the affinity moiety. In such circumstances, it may prove that the detection of a single epitope is not sufficient for conclusive identification of a target. Measuring the level of 2, preferably 3, more preferably 4 and even more preferably 5 or more markers within a single target, provides an unambiguous profile specific for the target of interest.

In another exemplary embodiment, the present invention provides a method for distinguishing between organisms expressing the same surface markers. Using SAC², it is possible to identify differences in targets based on the ratio of surface marker expression. For example, despite intense efforts, no single binding-receptor has been found for the unambiguous detection of B. anthracis spores, due to extensive cross-reactivity with related B. cereus and B. thuringiensis, which are genetically a single species (Helgason et al., Appl. Envir. Microbiol. 66:2627-2630 (2000)). Despite being of the same species, however, the relative amount of various surface proteins is different between the three bacilli. Thus, multi-point detection of a variety of markers at the single cell level will provide the specificity required to detect B. anthracis.

Diagnostic tests that detect the presence or absence of a single marker are unable to distinguish among strains that are nearly identical at the genetic level, highlighting the need for new tools to distinguish between closely related organisms. Epidemics caused by emerging variants of known pathogens are a common theme in infectious diseases (Jiang et al, Appl Environ Microbiol 66:148-53 (2000)) (Hedelberg et al. Nature 406:477-483 (2000)). There is also the problem of deliberate engineering of pathogens, incorporating virulence determinants from other species. An attack by such pathogens would be misdiagnosed due to the presence of markers not normally found in the host. By probing multiple markers within a single organism, using the methods of the invention, such variants are detected and preferably identified.

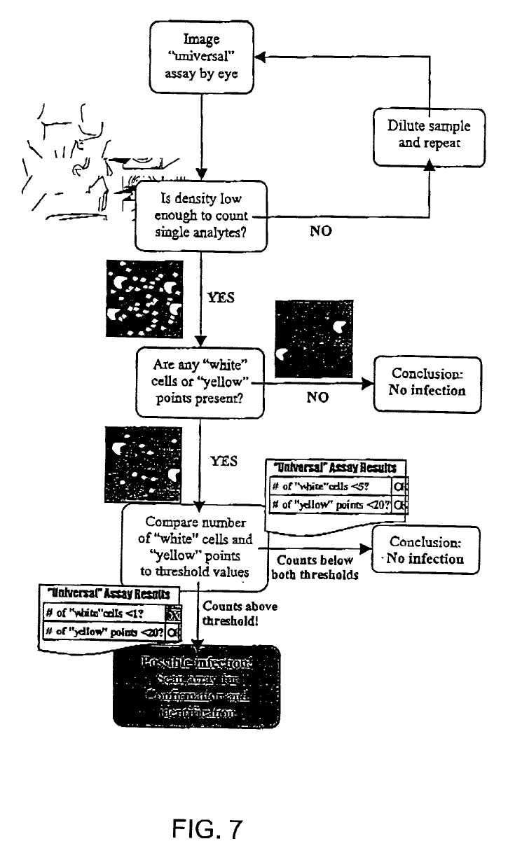

Detection by eye is also useful in those embodiments of the invention relying on SAC² (FIG. 7). The human eye is extremely good at distinguishing between subtly different combinations of colors, especially when the colors are chosen correctly. By way of illustration, it is trivial for people to distinguish between the colors red, green and yellow. Yellow, however, is simply the spectral sum of red and green, so if red and green quantum dots are used for molecular coincidence staining, positive assay signal can easily be identified by the perceived color, yellow. Other color combinations of use in this embodiment of the invention will be readily apparent to those of skill in the art, such as combinations of red, green and blue to form white.

In those embodiments of the invention in which multiple colors or ratiometric encoding are preferred for detection of the target, the creation of “white” light is preferably relied upon. Combinations of 3 and 4 colors can easily be chosen to produce white with fairly sensitive intensity dependence for each individual color. By controlling assay factors such as binding affinity, quantum yield and the number of quantum dots per receptor, differences in expression of surface proteins can be normalized so that the binding profile of the pathogenic organism of interest results in white emission while all other organisms preferably appear to be a non-white mixture of colors.

In another exemplary embodiment, SAC² is used to probe a solution-based assay. In this embodiment, the affinity moiety and the target species are labeled with different color quantum dots. Thus, a target-affinity moiety complex will include two quantum dots of different color. Using a technique such as confocal microscopy, it is possible to distinguish the bi-colored target-affinity moiety complex from the uncomplexed target and affinity moiety by the coincidence of two colors within a defined detection region of interest. Alternatively, as described above, the two colors of quantum dots can produce a third color, which is different from the color of the quantum dots attached, either directly or indirectly, to either the affinity moiety or the target. Detection of the third color within the region of interest confirms the presence of the target-affinity moiety complex. Alternatively, the second quantum dot color can be attached, either directly or indirectly, to the target-affinity moiety complex via a third labeled component such as an additional binding moiety, specific for either the target, the affinity moiety, the target-affinity moiety complex of any combination thereof.

In yet another exemplary embodiment, the application of SAC² to a particular assay results in an increase in the sensitivity of that assay to a level that is higher than the sensitivity of the assay using a quantum dot of a single color. The increase in sensitivity is realized in one or more detection regimes selected from ensemble detection, single target detection and detection in the transition regime. In this embodiment, sensitivity is improved by using coincidence signals as described above to differentiate specific from nonspecific signal, thereby allowing us to quantitatively detect target concentrations below the “intrinsic” nonspecific signal limit.

In yet another exemplary embodiment, different target species bound within the same assay region can be identified and differentiated from each other and from nonspecific signal by labeling the different target species with different combinations of quantum dot colors, and using those combinations of colors to identify the specific targets, as well as nonspecific signal.

D. Detection

Single molecule fluorescence detection can be achieved using a number of detection systems. The choice of a proper detection system for a particular application is well within the abilities of one of skill in the art. Exemplary detection means include, but are not limited to, detection by unaided eye, light microscopy using the eye or an optical sensor as the detector, confocal microscopy, laser scanning confocal microscopy, imaging using quantum dot color, fluorescence spectrum or other quantum dot property and wide-field imaging with a 2D CCD camera.

Once labeled, the fluorescence from the sample is detected. If the density of bound target molecules is from about 1 target/μm² to about 10⁶ target/μm², preferably from about 10 target/μm² to about 1 target/μm² then the assay signal is preferably measured and calibrated using the total emission intensity from the entire assay region (“ensemble counting”). If the target density is from about 10⁻⁵ target/μm² to about 1 target/μm² so that individual target molecules can be spatially resolved using standard far-field optics, then the assay signal is preferably measured and calibrated by counting the total number of bound target molecules (“single target counting”). The assay signal can be measured from all assays and assay regions using both ensemble and single target counting methods. A calibration curve can then be used to identify which assays fall in the ensemble regime, single target counting regime and transition regime.

In an exemplary embodiment, the detection system is capable of detecting the fluorescence from single semiconductor nanocrystals over the entire area of a 100 μm diameter assay region, with a spatial resolution of less than 0.5 μm. A preferred system uses a 2-dimensional CCD camera with a dynamic range of 65,536 counts per pixel and a read noise of ˜2 counts/pixel. If excitation intensity and integration time are selected to yield 30 counts/pixel/semiconductor nanocrystal, then in the single copy counting regime, individual semiconductor nanocrystals are detected with a signal to noise ratio of ˜15. Assuming an even distribution of bound molecules and a spatial resolution of ˜0.5 μm, up to about 40,000 individual spots within each 100 μm assay region can be detected. In an ideal system, this would result in a dynamic range within the single target counting regime of more than 10⁴. As the concentration increases into the ensemble regime, the average intensity increases linearly with concentration. The detector then provides an additional dynamic range of 10³ before saturating. As a result, a total dynamic range of 10⁷ is theoretically possible in a single experiment. In a preferred embodiment, multiple integration times are used to extend the dynamic range to higher concentrations if necessary.

In presently preferred embodiments, the detection method used to probe the assay resolves the fluorescence from a quantum dot associated with a single copy of a target species from the fluorescence arising from other quantum dots and from other fluorescence sources. For example, the probing method resolves a quantum dot attached, either directly or indirectly, to a selected single copy of a target species from other quantum dots attached, either directly or indirectly, to other single copies of the target in a population of labeled single copies of the target. As such, a necessary requirement for single target counting is that the spatial resolution limit of the detection system be sufficiently high to allow the detection of the labeled target molecules with less than 1 target molecules per resolution limited volume. For example, if the density of target molecules on an assay substrate were less than ˜1 molecule per μm², the spatial resolution of the detection system would need to be at least 1 μm in order to resolve the individual targets. If the density were 1 molecule per 100 μm², the spatial resolution of the detection system could potentially be decreased by a factor of 10×and still allow for single target detection Preferably this resolution limit should be ˜1 μm, although it would be possible in some cases to detect single targets using much lower resolution. In addition to spatial resolution, emission wavelength can also be used to resolve individual target molecules. If, for instance, different target molecules were labeled with 3 substantially non-overlapping colors of quantum dots, it is only necessary for the resolution limit of the detection system to allow the detection of labeled target molecules with less than 1 target molecule per resolution limited volume per color. This would allow either the resolution limit to be decreased by a factor of 3 or the concentration limit at the high concentration-end of the single target counting regime to be increased by a factor of three. Additional colors would further decrease the required spatial-resolution to target density ratio for single target counting.

In a presently preferred embodiment, the methods of the invention rely on wide-field imaging. By precisely controlling a scanning stage, taking multiple images of the field and stitching the images together, a larger region can be detected and quantified. Using this method, an entire 10000 element microarray can be scanned in less than 20 minutes using this invention.

In another preferred embodiment, the assay is probed with an optical detection system capable of detecting the fluorescence from single semiconductor nanocrystals (or other labels) with a spatial resolution of about 10 μm or less, preferably about 1 μm or less. In an exemplary embodiment, the optical system includes a wide-field imaging system with a 2D CCD camera and a high numerical aperture microscope objective. An exemplary laser based microscope system capable of detecting and spectrally resolving the fluorescence from single semiconductor nanocrystals is known in the art. The optical design of the above-referenced system is based on a wide-field epifluorescence microscope. FIG. 5 is a schematic drawing of the significant optical components. Excitation light from a laser source (488 nm Ar⁺) is transmitted through a 500 nm short pass dichroic mirror at an angle of 45°. The excitation light is then focused by a high numerical aperture microscope objective onto the sample surface. An additional lens optionally added to the excitation path causes collimated laser light to illuminate a wide area of the sample surface. The fluorescent image is collected by the same objective lens. The image is reflected by the dichroic mirror, passes through a wavelength-specific filter to remove any excitation light, and is focused by a final lens onto the detection system. The detection system consists of a 2D CCD camera and a tunable bandpass filter. Spectral images are obtained by acquiring multiple images each at a different wavelength. With this system, it is possible to simultaneously obtain spectra at every point within the image with a spectral resolution of 2 nm and a spatial resolution of less than ˜0.5 μm. Uniform excitation intensity in this system can be generated either through the use of a lamp light source or a laser excitation source that has been transformed from a Gaussian intensity profile to a “top-hat” profile through the use of a series of 2 Powel lenses, each oriented at 90 degrees relative to each other. Alternatively, the optical system can be comprised of a scanning confocal microscope system with a spatial resolution of less than 10 μm, preferably less than 1 μm and more preferably less than 0.5 μm.

In yet another exemplary embodiment, the optical system includes a microscope with an immersion microscope objective in which the sample is viewed from the backside of the sample substrate. The sample is located on the surface of the substrate and is detected with a high numerical aperture oil-immersion microscope and index matching immersion oil (e.g. refractive index=1.51). Using a system of this design can yield an increase in collection efficiency of as much as 800%. Alternatively, detection can be with a water-or other fluid-immersion lens, or a solid immersion lens (Mansfield, Stanford University Graduate Thesis, 1992) also detecting from the back-side of the sample substrate.

For ultrasensitive detection of single target molecules, it is preferred to have both a bright fluorophore and to minimize the collection of background fluorescence from the substrate surface and assay materials. In a preferred embodiment, autofluorescence from the assay substrate and assay materials is minimized by: (a) using low fluorescence array substrates such as quartz or low fluorescence glass; (b) choosing a fluorescent label that does not overlap significantly with the autofluorescence from the substrate and assay materials; and (c) choosing an excitation wavelength that does not significantly excite autofluorescence. Since semiconductor nanocrystals can be synthesized to absorb and emit at any wavelengths, they are a preferred fluorophore for minimizing interference from auto fluorescence.

Of concern in detecting labeled species on the single target counting level is how to locate assay regions with very low signal. For instance, if a microarray is labeled at a density in the single target counting regime, it may be difficult to locate the array spots for quantitative detection. In a preferred embodiment, kinematic alignment of the array slide combined with the use of “alignment spots” is used to locate the edges of the array and register the first image automatically so that the array spots are each located within the center of each image. Alignment spots are array spots with affinity moieties that are not specific for any target of interest. In an exemplary embodiment, a labeled species that is specific for these alignment spots is added at a known concentration to one or more assay mixtures. The alignment spots will, therefore, have a high signal and can be detected and used for alignment purposes. A pattern of alignment spots can be placed across each array that will unambiguously identify the absolute position of the array. Software can then be used to locate and analyze each spot within the array. Using pattern recognition algorithms, the alignment spots are identified and all other spot locations are determined from the known periodicity of the array. Once the array pattern is determined, each spot on the array can be located according to its position within a periodic lattice. The radius of all spots is preferably substantially the same and can be predetermined or extracted from the radius of the alignment spots.

Alternatively, a unique alignment affinity moiety can be added at a known concentration to every spot, and a unique alignment target, labeled with a quantum dot color that does not interfere with the detection methods described herein, can be added in a known amount to the sample solution. In this way, the boundaries of each assay region can be directly imaged.

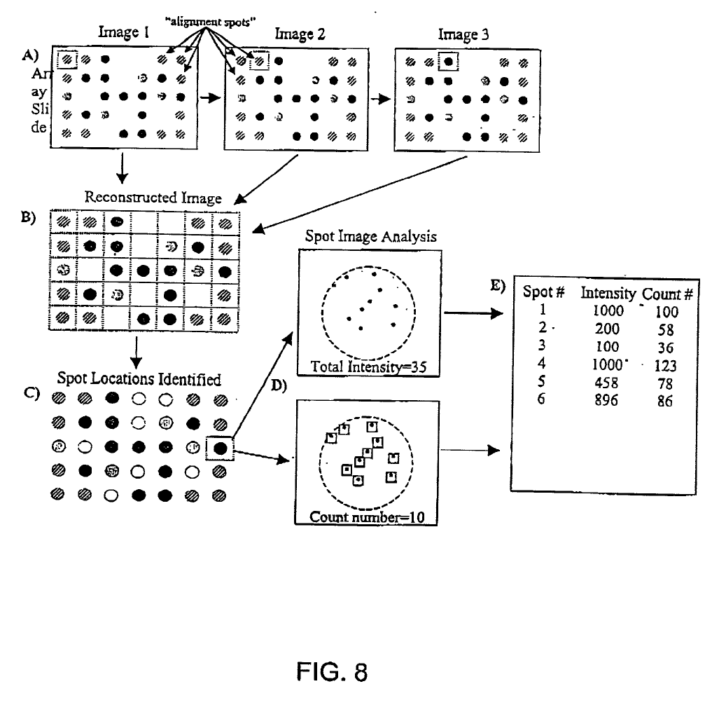

Two separate algorithms can then be used to analyze the signal from within each spot area. First, the total integrated signal from within each spot is measured and compared to either an equivalent area outside of the array spot or to a calibration spot of known intensity. Second, an algorithm is used to count individual fluorescent points within each array spot. Using pattern recognition, the algorithm will identify and count fluorescent points that fit a set of predetermined characteristics of shape, size and threshold intensity that are specific for the fluorescence from single semiconductor nanocrystals. For example, spots may be restricted to those that are the size and shape of the resolution limit of the detection system and of an intensity consistent with a fluorescent label detected with the particular detection system used. A data file is exported containing the ensemble intensity and the “count number” (i.e. the number of discrete fluorescent points) for each spot. FIG. 8 illustrates an exemplary complete array scanning procedure.

For some surface-based assays such as microtitre plate assays, macroscopic alignment of the optical system is preferably used (i.e. scanning the entire bottom of each microtitre well). For bead-based assays, it is preferred to use a second semiconductor nanocrystal color that does not spectrally overlap with the detection label. This second color can be added to each bead, either internally, or bound to the surface at a known concentration. This color can then be used to locate individual beads. Once found, a bandpass filter can be used to block the fluorescence from the alignment color and allow single target detection of only the label semiconductor nanocrystals. This 2-color technique can also be used for microarrays, microtitre-plate-based assays or any other surface-based assay.

One additional feature preferred for an assay system capable of detecting single bound target molecules is the elimination of nonspecific binding of the detection label to prevent interference by non-specifically bound fluorophores with the quantitative measure of target concentrations on the level of single target counting. In a preferred embodiment, labeling of these assays will be with a fluorophore with extremely low nonspecific binding. Preliminary results indicate that semiconductor nanocrystals show extremely low levels of nonspecific binding on printed cDNA microarrays and other assay substrates such as nitrocellulose. In addition, because the surface of semiconductor nanocrystals can be modified to have virtually any functionality, it is possible to continually tune the surface characteristics to minimize nonspecific binding.

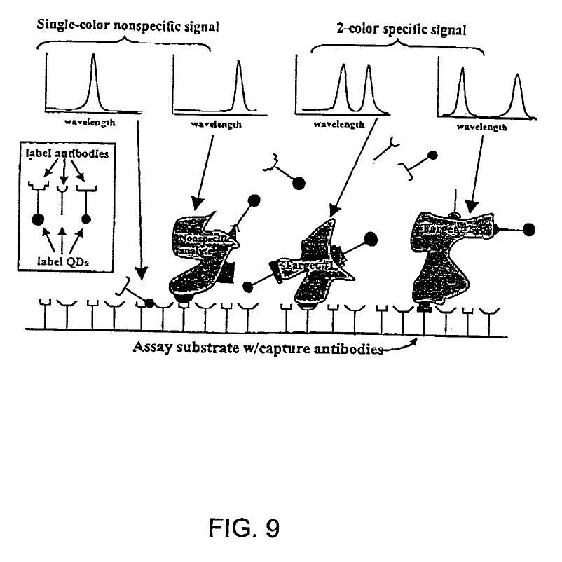

In an alternative embodiment, each target molecule can be labeled with two or more different semiconductor nanocrystal colors via two or more different binding interactions. Specifically bound labels can then be identified through the detection of both colors colocalized within the same fluorescent spot. Nonspecific binding is identified by single color fluorescence (FIG. 9). See, section C, supra.

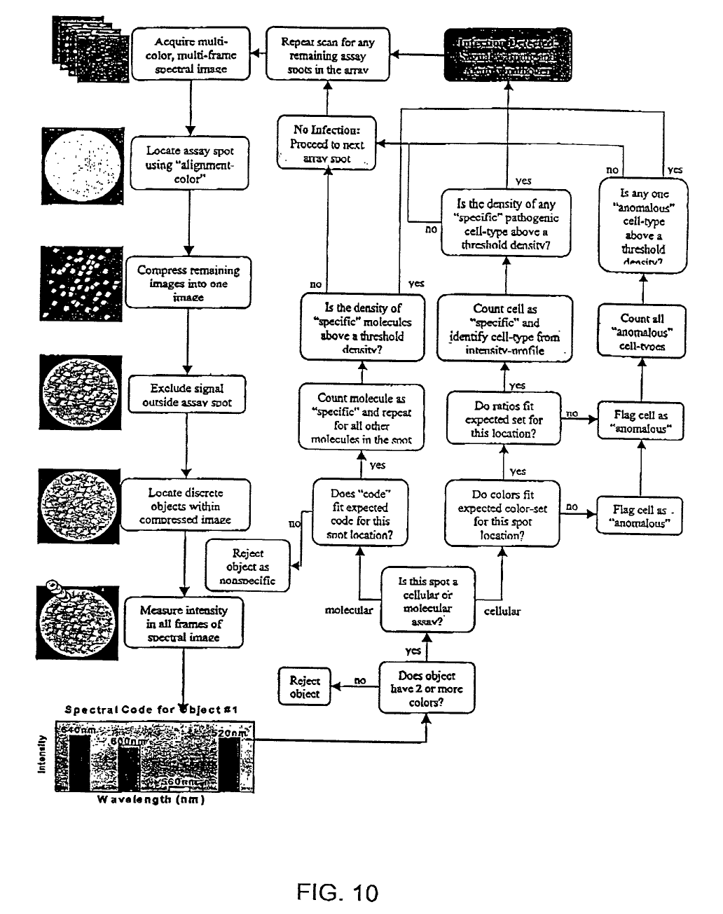

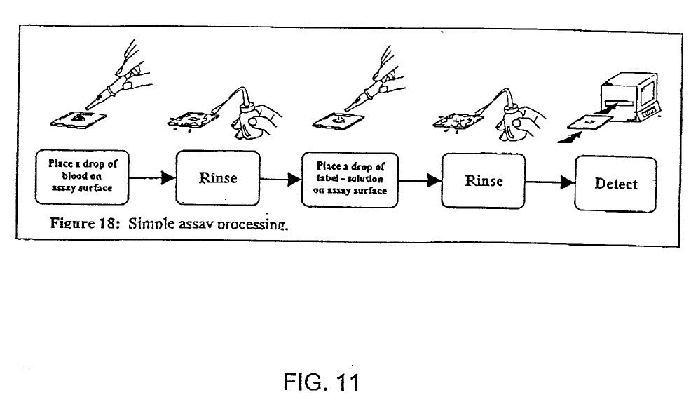

The data acquired from the assay is preferably processed using algorithms for image-and data-analysis. An exemplary algorithm is shown in FIG. 10. An exemplary method for SAC² detected ‘by eye’ is shown in FIG. 7.

E. Substrates

In an exemplary embodiment, an assay of the invention is performed on a surface support such as a microarray substrate, the bottom of a microtitre plate or a polymer bead. The assay can be any assay that utilizes optical detection such as fluorescence or light scattering to quantitate the assay signal. This includes, but is not limited to, DNA or RNA hybridization assays, fluorescence in situ hybridization (FISH), immunoassays, and molecular beacon assays. One or more assay components can be labeled with a semiconductor nanocrystal and/or other fluorophore such as an organic dye or metal colloid. The assay can be either directly or indirectly labeled. In a presently preferred embodiment, the assay of the invention utilizes direct or indirect labeling of one or more assay components in which semiconductor nanocrystals are used as the label. Semiconductor nanocrystals can be incorporated into the assay via a plurality of techniques well known in the art. Each bound target molecule is labeled with one or more semiconductor nanocrystals.

In the single target detection method of the invention, the affinity moiety for the target is immobilized on a substrate, either directly or through a spacer arm that is intercalated between the substrate and the affinity moiety. Alternatively, the affinity moiety is contained within a structure on the substrate (e.g., a well, trough, etc.). Substrates that are useful in practicing the present invention can be made of any stable material, or combination of materials. Moreover, useful substrates can be configured to have any convenient geometry or combination of structural features. The substrates can be either rigid or flexible and can be either optically transparent or optically opaque. The substrates can also be electrical insulators, conductors or semiconductors. Further the substrates can be substantially impermeable to liquids, vapors and/or gases or, alternatively, the substrates can be substantially permeable to one or more of these classes of materials.

The materials forming the substrate are utilized in a variety of physical forms such as films, supported powders, glasses, crystals and the like. For example, a substrate can consist of a single inorganic oxide or a composite of more than one inorganic oxide. When more than one component is used to form a substrate, the components can be assembled in, for example a layered structure (i.e., a second oxide deposited on a first oxide) or two or more components can be arranged in a contiguous non-layered structure. In addition, one or more components can be admixed as particles of various sizes and deposited on a support, such as a glass, quartz or metal sheet. Further, a layer of one or more components can be intercalated between two other substrate layers (e.g., metal-oxide-metal, metal-oxide-crystal). Those of skill in the art are able to select an appropriately configured substrate, manufactured from an appropriate material for a particular application.

Exemplary substrate materials include, but are not limited to, inorganic crystals, inorganic glasses, inorganic oxides, metals, organic polymers and combinations thereof. Inorganic glasses and crystals of use in the substrate include, but are not limited to, LiF, NaF, NaCl, KBr, KI, CaF₂, MgF₂, HgF₂, BN, AsS₃, ZnS, Si₃N₄ and the like. The crystals and glasses can be prepared by art standard techniques. See, for example, Goodman, CRYSTAL GROWTH THEORY AND TECHNIQUES, Plenum Press, New York 1974. Alternatively, the crystals can be purchased commercially (e.g., Fischer Scientific). Inorganic oxides of use in the present invention include, but are not limited to, Cs₂O, Mg(OH)₂, TiO₂, ZrO₂, CeO₂, Y₂O₃, Cr₂O₃, Fe₂O₃, NiO, ZnO, Al₂O₃, SiO₂ (glass), quartz, In₂O₃, PbO₂, and the like. Metals of use in the substrates of the invention include, but are not limited to, gold, silver, platinum, palladium, nickel, copper and alloys and composites of these metals.

Organic polymers that form useful substrates include, for example, polyalkenes (e.g., polyethylene, polyisobutene, polybutadiene), polyacrylics (e.g., polyacrylate, polymethyl methacrylate, polycyanoacrylate), polyvinyls (e.g., polyvinyl alcohol, polyvinyl acetate, polyvinyl butyral, polyvinyl chloride), polystyrenes, polycarbonates, polyesters, polyurethanes, polyamides, polyimides, polysulfone, polysiloxanes, polyheterocycles, cellulose derivative (e.g., methyl cellulose, cellulose acetate, nitrocellulose), polysilanes, fluorinated polymers, epoxies, polyethers and phenolic resins.

In a preferred embodiment, the substrate material is substantially non-reactive with the target, thus preventing non-specific binding between the substrate and the target or other components of an assay mixture. Methods of coating substrates with materials to prevent non-specific binding are generally known in the art. Exemplary coating agents include, but are not limited to cellulose, bovine serum albumin, and poly(ethyleneglycol). The proper coating agent for a particular application will be apparent to one of skill in the art.

In a further preferred embodiment, the substrate material is substantially non-fluorescent or emits light of a wavelength range that does not interfere with the detection of the target. Exemplary low-background substrates include those disclosed by Cassin et al, U.S. Pat. No. 5,910,287 and Pham et al., U.S. Pat. No. 6,063,338.

The surface of a substrate of use in practicing the present invention can be smooth, rough and/or patterned. The surface can be engineered by the use of mechanical and/or chemical techniques. For example, the surface can be roughened or patterned by rubbing, etching, grooving, stretching, and the oblique deposition of metal films. The substrate can be patterned using techniques such as photolithography (Kleinfield et al., J Neurosci. 8: 4098-120 (1998)), photoetching, chemical etching and microcontact printing (Kumar et al, Langmuir 10: 1498-511 (1994)). Other techniques for forming patterns on a substrate will be readily apparent to those of skill in the art.

The affinity moiety is generally immobilized on the substrate. When the affinity moiety is bound on the substrate, the binding is typically between a functional group presented by the surface of the substrate and a complementary functional group on the affinity moiety. Alternatively, the interaction is between a functional group on a spacer arm that links the substrate and the affinity moiety.

Currently favored classes of reactions for coupling an affinity moiety to a reactive spacer proceed under relatively mild conditions. These include, but are not limited to nucleophilic substitutions (e.g., reactions of amines and alcohols with acyl halides), electrophilic substitutions (e.g., enamine reactions) and additions to carbon-carbon and carbon-heteroatom multiple bonds (e.g., Michael reaction, Diels-Alder addition). These and other useful reactions are discussed in March, J., ADVANCED ORGANIC CHEMISTRY, Third Ed., John Wiley & Sons, New York, 1985.

According to an exemplary embodiment, a substrate's surface is functionalized with one or more distinct spacer arms by covalently binding a reactive spacer arm to the substrate surface in such a way as to derivatize the substrate surface with a plurality of available reactive functional groups presented by the spacer arm. Preferred reactive groups include, for example, amines, hydroxyl groups, carboxylic acids, carboxylic acid derivatives, alkenes, sulfhydryls, siloxanes, and the like

A number of reaction types are available for the functionalization of a substrate surface. For example, substrates constructed of a plastic such as polypropylene, can be surface derivatized by chromic acid oxidation, and subsequently converted to hydroxylated or aminomethylated surfaces. Substrates made from highly crosslinked divinylbenzene can be surface derivatized by chloromethylation and subsequent functional group manipulation. Additionally, functionalized substrates can be made from etched, reduced poly-tetrafluoroethylene. Other methods of derivatizing polymeric substrates are known to those of skill in the art.

When the substrates are constructed of a siliceous material such as glass or quartz, the surface can be derivatized by reacting the surface Si—OH, SiO—H, and/or Si—Si groups with a functionalizing reagent. In a preferred embodiment, wherein the substrates are made from glass, the covalent bonding of the reactive group to the substrate surface is achieved by conversion of groups on the substrate surface by a silicon-modifying reagent such as:

(R¹O)₃—Si—R²—X¹

in which R¹ is typically an alkyl group, such as methyl or ethyl, R² is a linking group, such as alkylene or heteroalkylene, between silicon and X¹. X¹ represents a reactive group or a protected reactive group. The reactive group can also be an affinity moiety. Silane derivatives having halogens or other leaving groups beside the displayed alkoxy groups are also useful in the present invention.

A number of siloxane functionalizing reagents can be used to form substrates of use in the present invention. Representative reagent include:

1. Hydroxyalkyl siloxanes (silylate surface, functionalize with diborane, and H₂O₂ to oxidize the alcohol)

a. allyl trichlorosilane→→3-hydroxypropyl

b. 7-oct-1-enyl trichlorchlorosilane→→8-hydroxyoctyl

2. Diol (dihydroxyalkyl) siloxanes (silylate surface and hydrolyze to diol)

a. (glycidyl trimethoxysilane→→(2,3-dihydroxypropyloxy)propyl

3. Aminoalkyl siloxanes (amines requiring no intermediate functionalizing step)

a. 3-aminopropyl trimethoxysilane→aminopropyl

4. Dimeric secondary aminoalkyl siloxanes

a. bis-(3-trimethoxysilylpropyl) amine→bis(silyloxylpropyl)amine.

It will be apparent to those of skill in the art that an array of similarly useful functionalizing chemistries are available when spacer arms other than siloxanes are used. For example similarly functionalized alkyl thiols can be attached to metal films and subsequently reacted to produce the functional groups such as those exemplified above

In another preferred embodiment, the functionalizing reagent provides more than one reactive group per each reagent molecule. Using reagents such as that exemplified below, each reactive site on the substrate surface is, in essence, “amplified” to two or more functional groups:

(R¹O)₃—Si—R²—(X¹)ₙ

wherein, R, R² and X¹ are as described above. The letter n represents an integer between about 2 and about 50, and more preferably between about 2 and about 20.

The linker group R² is selected from groups that are stable or they can be cleaved by chemical reactions induced by, for example, heat, light, cleaving reagents, electrochemical reactions, etc. For example, R² groups comprising ester or disulfide bonds can be cleaved by hydrolysis and reduction, respectively. R² groups that are cleaved by light include, for example, nitrobenzyl derivatives, phenacyl groups, benzoin esters, etc. Many cleaveable groups are known in the art. See, for example, Jung et al., Biochem. Biophys. Acta, 761: 152-162(1983); Joshi etal., J. Biol. Chem., 265: 14518-14525(1990); Zarling et al., J. Immunol., 124: 913-920 (1980); Bouizar et al., Eur. J. Biochem., 155: 141-147 (1986); Park et al, J. Biol. Chem., 261: 205-210 (1986); Browning et al., J. Immunol., 143: 18591867 (1989).

Selection of an appropriate reactive functional group, X¹, for a particular application is well within the abilities of one of skill in the art. Presently preferred groups include:

(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups which can be converted to esters, ethers, aldehydes, etc.

(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups which can be converted to disulfides or reacted with acyl halides;

(h) amine or sulfhydryl groups which can be, for example, acylated or alkylated;

(i) alkenes which can undergo, for example, cycloadditions, acylation, Michael addition, etc; and

(j) epoxides which can react with, for example, amines and hydroxyl compounds.

X¹ can be chosen such that it does not participate in, or interfere with, the reaction controlling the attachment of the functionalized spacer component onto the substrate's surface. Alternatively, the reactive functional group can be protected from participating in the reaction by the presence of a protecting group. Those of skill in the art understand how to protect a particular functional group from interfering with a chosen set of reaction conditions. For examples of useful protecting groups, See Greene et al., PROTECTIVE GROUPS IN ORGANIC SYNTHESIS, John Wiley & Sons, New York, 1991.

In a preferred embodiment, the spacer arm bearing the affinity moiety is attached essentially irreversibly via a “stable bond” to the surface of the substrate. A “stable bond”, as used herein, is a bond, which maintains its chemical integrity over a wide range of conditions (e.g., amide, carbamate, carbon-carbon, ether, etc.). In another preferred embodiment the spacer arm bearing the affinity moiety is attached to the substrate surface by a “cleaveable bond”. A “cleaveable bond”, as used herein, is a bond which is designed to undergo scission under conditions which do not degrade other bonds in the affinity moiety-target complex. Cleaveable bonds include, but are not limited to, disulfide, imine, carbonate and ester bonds.

In addition to being used to tether the affinity moiety to the substrate, spacer arms, are used to control the physical and chemical properties of the substrate. Properties that are usefully controlled include, for example, hydrophobicity, hydrophilicity, surface-activity, non-specific binding and the distance of the affinity moiety from the plane of the substrate and/or the spacer arm.

The hydrophilicity of the substrate surface can be enhanced by reaction with polar molecules such as amine-, hydroxyl- and polyhydroxyl-containing molecules. Representative examples include, but are not limited to, polylysine, polyethyleneimine, poly(ethyleneglycol) and poly(propyleneglycol). Suitable functionalization chemistries and strategies for these compounds are known in the art. See, for example, Dunn, R. L., et al., Eds. POLYMERIC DRUGS AND DRUG DELIVERY SYSTEMS, ACS Symposium Series Vol. 469, American Chemical Society, Washington, D.C. 1991; Herren et al., J. Colloid and Interfacial Science 115: 46-55 (1987); Nashabeh et al., J. Chromatography 559: 367-383 (1991); Balachandar et al., Langmuir 6: 1621-1627 (1990); and Bums et al., Biomaterials 19: 423-440 (1998).

The hydrophobicity of the substrate surface can be modulated by using a hydrophobic spacer arm such as, for example, long chain diamines, long-chain thiols, α, o-amino acids, etc. Representative hydrophobic spacers include, but are not limited to, 1,6-hexanediamine, 1,8-octanediamine, 6-aminohexanoic acid and 8-aminooctanoic acid.

The substrate surface can also be made surface-active by attaching to the substrate surface a spacer that has surfactant properties.

In another embodiment, the spacer serves to distance the affinity moiety from the substrate. Spacer arms with this characteristic have several uses. For example, an affinity moiety held too closely to the substrate surface may not interact with incoming target, or it may react unacceptably slowly. When either or both the target or the affinity moiety are sterically demanding, the interaction leading to affinity moiety-target complex formation can be undesirably slowed, or not occur at all, due to the monolithic substrate hindering the approach of the two components.

In another embodiment, the physicochemical characteristics (e.g., hydrophobicity, hydrophilicity, surface activity, conformation) of the substrate surface and/or spacer arm are altered by attaching a monovalent moiety which is different in composition than the constituents of the spacer arm and which does not bear an affinity moiety. As used herein, “monovalent moiety” refers to organic molecules attached to the substrate that do not bear an affinity moiety. “Monovalent moieties” are to be contrasted with the “spacer” groups described above. Such monovalent groups are used to modify the hydrophilicity, hydrophobicity, binding characteristics, etc. of the substrate surface. Examples of groups useful for this purpose include long chain alcohols, amines, fatty acids, fatty acid derivatives, poly(ethyleneglycol), poly(ethyleneglycol)monoalkyl ethers, etc.

In an exemplary embodiment, those regions of the substrate that do not have bound thereto an affinity moiety or spacer-arm affinity moiety construct are “blocked” or “capped” by the use of a monovalent moiety that minimizes or prevents adventitious, nonspecific binding of assay mixture components to the substrate surface. A preferred monovalent moiety for this purpose is poly(ethylene glycol) and derivatives thereof. Alternative capping agents include, for example, blocking agents such as BSA (from 0-5% in PBS), commercial blocking buffers (e.g., Superblock) and common cocktails of proteins, serum and DNA-based blocking agents.

Polyethylene glycol (PEG) is used in biotechnology and biomedical applications. The use of this agent has been reviewed (POLY(ETHYLENE GLYCOL) CHEMISTRY: BIOTECHNICAL AND BIOMEDICAL APPLICATIONS, J. M. Harris, Ed., Plenum Press, New York, 1992). Modification of enzymes (Chiu et al., J. Bioconjugate Chem., 4: 290-295 (1993)), RGD peptides (Braatz et al., Bioconjugate Chem., 4: 262-267 (1993)), liposomes (Zalipsky, S. Bioconjugate Chem., 4: 296-299 (1993)), and CD4-IgG glycoprotein (Chamow et al, Bioconjugate Chem., 4: 133-140 (1993)) are some of the recent advances in the use of polyethylene glycol. Surfaces treated with PEG have been shown to resist protein deposition and have improved resistance to thrombogenicity when coated on blood contacting biomaterials (Merrill, “Poly(ethylene oxide) and Blood Contact: A Chronicle of One Laboratory,” in POLY(ETHYLENE GLYCOL) CHEMISTRY: BIOTECHNICAL AND BIOMEDICAL APPLICATIONS, Harris, Ed., Plenum Press, New York, (1992), pp. 199-220).

Many activated derivatives of poly(ethyleneglycol) are available commercially and in the literature. It is well within the abilities of one of skill to choose, and synthesize if necessary, an appropriate activated PEG derivative with which to prepare a substrate useful in the present invention. See, Abuchowski et al. Cancer Biochem. Biophys., 7: 175-186 (1984); Abuchowski et al., J. Biol. Chem., 252: 3582-3586 (1977); Jackson et al., Anal. Biochem., 165: 114-127 (1987); Koide et al., Biochem Biophys. Res. Commun., 111: 659-667 (1983)), tresylate (Nilsson et al., Methods Enzymol., 104: 56-69 (1984); Delgado et al, Biotechnol Appl. Biochem., 12: 119-128 (1990)); N-hydroxysuccinimide derived active esters (Buckmann et al., Makromol. Chem., 182: 1379-1384 (1981); Joppich et al., Makromol. Chem., 180: 1381-1384 (1979); Abuchowski et al., Cancer Biochem. Biophys., 7: 175-186 (1984); Katreet al. Proc. Aatl. Acad. Sci. U.S.A., 84: 1487-1491 (1987); Kitamura et al., Cancer Res., 51: 4310-4315 (1991); Boccu et al., Z. Naturforsch., 38C: 94-99 (1983), carbonates (Zalipsky et al., POLY(ETHYLENE GLYCOL) CHEMISTRY: BIOTECHNICAL AND BIOMEDICAL APPLICATIONS, Harris, Ed., Plenum Press, New York, 1992, pp. 347-370; Zalipsky et al., Biotechnol. Appl. Biochem., 15: 100-114 (1992); Veronese et al., Appl. Biochem. Biotech., 11: 141-152 (1985)), imidazolyl formates (Beauchamp et al., Anal. Biochem., 131: 25-33 (1983); Berger et al., Blood, 71: 1641-1647 (1988)), 4-dithiopyridines (Woghiren et al., Bioconjugate Chem., 4: 314-318 (1993)), isocyanates (Byun et al., ASAIO Journal, M649-M-653 (1992)) and epoxides (U.S. Pat. No. 4,806,595, issued to Noishiki et al., (1989). Other linking groups include the urethane linkage between amino groups and activated PEG. See, Veronese, et al., Appl. Biochem. Biotechnol., 11: 141-152 (1985).

The specificity and multiplexing capacity of the assays of the invention can be increased by incorporating spatial encoding (e.g., spotted microarrays) into the assay. Spatial encoding can be introduced into each of the assays of the invention. In an exemplary embodiment, capture antibodies for different analytes can be arrayed across the assay surface, allowing specific spectral codes (see, Sections B and C) to be reused in each location. In this case, the array location is an additional encoding parameter, allowing the detection of a virtually unlimited number of different analytes.

While a large number of targets can be detected simultaneously using a spatial array, the time involved to scan all array positions for all colors may limit the ease of use for larger arrays. To circumvent this problem, in a preferred embodiment, a spatially encoded array will include a rough, first level assay. The first level assay is preferably embodied in an array spot containing a mixture of all, or a selected population of the affinity moieties on the array. Multi-color signal in this spot indicates the presence of a captured target on the array, preferably prompting the system or user to scan the entire array for specific identification. The presence of a first level assay location on the spatial array significantly increases the ease and speed of the assay by only scanning samples containing a target.

In the embodiments of the invention in which spatial encoding is utilized, they utilize a spatially encoded array comprising m molecules or organisms (affinity moieties) distributed over m regions of a substrate. Each of the m affinity moieties is preferably a different moiety, although assays in which the same affinity moiety is located in two or more locations are within the scope of the present invention. The m affinity moieties are preferably patterned on the substrate in a manner that allows the identity of each of the m locations to be ascertained. In a preferred embodiment, the m affinity moieties are ordered in a p by q matrix of p×q discrete locations, wherein each of the p×q location has bound thereto at least one of the m affinity moieties. The microarray can be patterned from essentially any type of affinity moiety, including small organic molecules, peptides, nucleic acids, carbohydrates, antibodies, enzymes, cells and the like. In an exemplary embodiment, the affinity moieties are labeled with a quantum dot.

The spatially encoded assay substrates can include substantially any number of compounds. In a preferred embodiment, m is a number from 1 to 100, more preferably, from 10 to 1,000, and more preferably from 100 to 10,000.

A variety of methods are currently available for making arrays of biological macromolecules, such as arrays of antibodies, nucleic acid molecules or proteins. The following discussion utilizes a DNA microarray as an exemplary microassay. This use of DNA is intended to be illustrative and not limiting. Microarrays useful in practicing the present invention can be made with a wide range of other compound types.

One method for making ordered arrays of DNA on a substrate is a “dot blot” approach. In this method, a vacuum manifold transfers a plurality, e.g., 96, aqueous samples of DNA from 3 millimeter diameter wells to a porous membrane. A common variant of this procedure is a “slot-blot” method in which the wells have highly-elongated oval shapes.

The DNA is immobilized on the substrate by baking the membrane or exposing it to UV radiation. This is a manual procedure practical for making one array at a time and usually limited to 96 samples per array. “Dot-blot” procedures are therefore inadequate for applications in which many thousand samples must be determined.

A more efficient technique employed for making ordered arrays of genomic fragments uses an array of pins dipped into the wells, e.g., the 96 wells of a microtitre plate, for transferring an array of samples to a substrate, such as a porous membrane, glass surface, or the like. One array includes pins that are designed to spot a membrane in a staggered fashion, for creating an array of 9216 spots in a 22×22 cm area. See, Lehrach, et al., HYBRIDIZATION FINGERPRINTING IN GENOME MAPPING AND SEQUENCING, GENOME ANALYSIS, Vol. 1, Davies et al, Eds., Cold Springs Harbor Press, pp. 39-81 (1990).

An alternate method of creating ordered arrays of nucleic acid sequences is described by Pirrung et al. (U.S. Pat. No. 5,143,854, issued 1992), and also by Fodor et al., (Science, 251: 767-773 (1991)). The method involves synthesizing different nucleic acid sequences at different discrete regions of a particle. This method employs elaborate synthetic schemes, and is generally limited to relatively short nucleic acid sample, e.g., less than 20 bases. A related method has been described by Southern et al. (Genomics, 13: 1008-1017 (1992)).

Khrapko, et al., DNA Sequence, 1: 375-388 (1991) describes a method of making an oligonucleotide matrix by spotting DNA onto a thin layer of polyacrylamide. The spotting is done manually with a micropipette.

F. Affinity moieties

As used herein, the term “affinity moiety” refers to a species, which recognizes and interacts detectably with a target. An affinity moiety can be or can include any structure or combination of structures that allow it to interact with the target. Affinity moieties are preferably selected from organic functional groups, organometallic agents, inorganic materials, biomolecules, bioactive molecules, cells, and species that are combinations of two or more such elements.

In an exemplary embodiment, the affinity moiety comprises an organic functional group that interacts with a component of the target. In presently preferred embodiments, the organic functional group is selected from simple groups, such as amines, carboxylic acids, alcohols, sulfhydryls and the like. Functional groups presented by more complex species are also of use, such as those presented by drugs, chelating agents, crown ethers, cyclodextrins, and the like. In an exemplary embodiment, the affinity moiety is an amine that interacts with a structure on the target that binds to the amine (e.g., carbonyl groups, alkylhalo groups), or which protonates the amine (e.g., carboxylic acid, sulfonic acid) to form an ion pair. In another exemplary embodiment, the affinity moiety is a carboxylic acid, which interacts with the target by complexation (e.g., metal ions), or which protonate a basic group on the target (e.g. amine) forming an ion pair.

The organic functional group can be a component of a small organic molecule with the ability to specifically recognize a target molecule. Exemplary small organic molecules include, but are not limited to, amino acids, biotins, carbohydrates, glutathiones, and nucleic acids.

Typical amino acids suitable as affinity ligands include L-alanine, L-arginine, L-asparagine, L-aspartic acid, L-cysteine, L-cystine, L-glutamic acid, L-glutamine, glycine, L-histidine, L-isoleucine, L-lysine, L-methionine, L-phenylalanine, L-proline, L-serine, L-threonine, L-thyroxine, D-tryptophan, L-tryptophan, L-tyrosine and L-valine. Typical avidinbiotin ligands include avidin, biotin, desthiobiotin, diaminobiotin, and 2-iminobiotin. Typical carbohydrates include glucoseamines, glycopryranoses, galactoseamines, the fucosamines, the fucopyranosylamines, the galactosylamines, the glycopyranosides, and the like. Typical glutathione ligands include glutathione, hexylglutathione, and sulfobromophthalein-S-glutathione.

In another exemplary embodiment, the affinity moiety is a biomolecule, such as a natural or synthetic peptide, antibody, nucleic acid, saccharide, lectin, receptor, antigen, cell or a combination thereof. Thus, in an exemplary embodiment, the affinity moiety is an antibody raised against a target or against a species that is structurally analogous to a target. In another exemplary embodiment, the affinity moiety is avidin, or a derivative thereof, which binds to a biotinylated analogue of the target. In still another exemplary embodiment, the affinity moiety is a nucleic acid, which binds to single- or double-stranded nucleic acid target having a sequence complementary to that of the affinity moiety.

Biomolecules useful in practicing the present invention are derived from any source. The biomolecules can be isolated from natural sources or can be produced by synthetic methods. Proteins can be natural proteins, mutated proteins or fusion proteins. Mutations can be effected by chemical mutagenesis, site-directed mutagenesis or other means of inducing mutations known to those of skill in the art. Proteins useful in practicing the instant invention include, for example, enzymes, antigens, antibodies and receptors. Antibodies can be either polyclonal or monoclonal.

Affinity moieties, which are antibodies can be used to recognize targets which include, but are not limited to, proteins, peptides, nucleic acids, saccharides or small molecules such as drugs, herbicides, pesticides, industrial chemicals, organisms, cells and agents of war. Methods of raising antibodies against specific molecules or organisms are well-known to those of skill in the art. See, U.S. Pat. No. 5/147,786, issued to Feng et al. on Sep. 15, 1992; No. 5/334,528, issued to Stanker et al. on Aug. 2, 1994; No. 5/686,237, issued to Al-Bayati, M.A.S. on Nov. 11, 1997; and No. 5/573,922, issued to Hoess et al. on Nov. 12, 1996.

Antibodies and other peptides can be attached to a substrate or spacer arm by any available reactive group. For example, peptides can be attached through an amine, carboxyl, sulfhydryl, or hydroxyl group. Such a group can reside at a peptide terminus or at a site internal to the peptide chain. The peptide chains can be further derivatized at one or more sites to allow for the attachment of appropriate reactive groups onto the chain. See, Chrisey et al Nucleic Acids Res. 24:3031-3039 (1996). Methods for attaching antibodies to surfaces are also known in the art. See, Delamarche et al. Langmuir 12:1944-1946 (1996).

In another exemplary embodiment, the affinity moiety is a drug moiety. The drug moieties can be agents already accepted for clinical use or they can be drugs whose use is experimental, or whose activity or mechanism of action is under investigation. The drug moieties can have a proven action in a given disease state or can be only hypothesized to show desirable action in a given disease state. In a preferred embodiment, the drug moieties are compounds which are being screened for their ability to interact with a target of choice. As such, drug moieties which are useful in practicing the instant invention include drugs from a broad range of drug classes having a variety of pharmacological activities.

Exemplary classes of useful agents include, but are not limited to, non-steroidal anti-inflammatory drugs (NSAIDS). The NSAIDS can, for example, be selected from the following categories: (e.g., propionic acid derivatives, acetic acid derivatives, fenamic acid derivatives, biphenylcarboxylic acid derivatives and oxicams); steroidal anti-inflammatory drugs including hydrocortisone and the like; antihistaminic drugs (e.g., chlorpheniramine, triprolidine); antitussive drugs (e.g., dextromethorphan, codeine, carmiphen and carbetapentane); antipruritic drugs (e.g., methidilizine and trimeprizine); anticholinergic drugs (e.g., scopolamine, atropine, homatropine, levodopa); anti-emetic and antinauseant drugs (e.g., cyclizine, meclizine, chlorpromazine, buclizine); anorexic drugs (e.g., benzphetamine, phentermine, chlorphentermine, fenfluramine); central stimulant drugs (e.g., amphetamine, methamphetamine, dextroamphetamine and methylphenidate); antiarrhythmic drugs (e.g., propanolol, procainamide, disopyraminde, quinidine, encainide); β-adrenergic blocker drugs (e.g., metoprolol, acebutolol, betaxolol, labetalol and timolol); cardiotonic drugs (e.g., milrinone, amrinone and dobutamine); antihypertensive drugs (e.g., enalapril, clonidine, hydralazine, minoxidil, guanadrel, guanethidine);diuretic drugs (e.g., amiloride and hydrochlorothiazide); vasodilator drugs (e.g., diltazem, amiodarone, isosuprine, nylidrin, tolazoline and verapamil); vasoconstrictor drugs (e.g., dihydroergotamine, ergotamine and methylsergide); antiulcer drugs (e.g., ranitidine and cimetidine); anesthetic drugs (e.g., lidocaine, bupivacaine, chlorprocaine, dibucaine); antidepressant drugs (e.g., imipramine, desipramine, amitryptiline, nortryptiline); tranquilizer and sedative drugs (e.g., chlordiazepoxide, benacytyzine, benzquinamide, flurazapam, hydroxyzine, loxapine and promazine); antipsychotic drugs (e.g., chlorprothixene, fluphenazine, haloperidol, molindone, thioridazine and trifluoperazine); antimicrobial drugs (antibacterial, antifungal, antiprotozoal and antiviral drugs).

Antimicrobial drugs which are preferred for incorporation into the present composition include, for example, pharmaceutically acceptable salts of β-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, hexamidine isothionate, metronidazole, pentamidine, gentamycin, kanamycin, lineomycin, methacycline, methenamine, minocycline, neomycin, netilmycin, paromomycin, streptomycin, tobramycin, miconazole and amanfadine.

Other drug moieties of use in practicing the present invention include antineoplastic drugs (e.g., antiandrogens (e.g., leuprolide or flutamide), cytocidal agents (e.g., adriamycin, doxorubicin, taxol, cyclophosphamide, busulfan, cisplatin, α-2-interferon) anti-estrogens (e.g., tamoxifen), antimetabolites (e.g., fluorouracil, methotrexate, mercaptopurine, thioguanine).

The affinity moiety can also comprise hormones (e.g., medroxyprogesterone, estradiol, leuprolide, megestrol, octreotide or somatostatin); muscle relaxant drugs (e.g., cinnamedrine, cyclobenzaprine, flavoxate, orphenadrine, papaverine, mebeverine, idaverine, ritodrine, dephenoxylate, dantrolene and azumolen); antispasmodic drugs; bone-active drugs (e.g., diphosphonate and phosphonoalkylphosphinate drug compounds); endocrine modulating drugs (e.g., contraceptives (e.g., ethinodiol, ethinyl estradiol, norethindrone, mestranol, desogestrel, medroxyprogesterone), modulators of diabetes (e.g., glyburide or chlorpropamide), anabolics, such as testolactone or stanozolol, androgens (e.g., methyltestosterone, testosterone or fluoxymesterone), antidiuretics (e.g., desmopressin) and calcitonins).

Also of use in the present invention are estrogens (e.g., diethylstilbesterol), glucocorticoids (e.g., triamcinolone, betamethasone, etc.) and progenstogens, such as norethindrone, ethynodiol, norethindrone, levonorgestrel; thyroid agents (e.g., liothyronine or levothyroxine) or anti-thyroid agents (e.g., methimazole); antihyperprolactinemic drugs (e.g., cabergoline); hormone suppressors (e.g., danazol or goserelin), oxytocics (e.g., methylergonovine or oxytocin) and prostaglandins, such as mioprostol, alprostadil or dinoprostone, can also be employed.

Other useful affinity moieties include immunomodulating drugs (e.g., antihistamines, mast cell stabilizers, such as lodoxamide and/or cromolyn, steroids (e.g., triamcinolone, beclomethazone, cortisone, dexamethasone, prednisolone, methylprednisolone, beclomethasone, or clobetasol), histamine H₂ antagonists (e.g., famotidine, cimetidine, ranitidine), immunosuppressants (e.g., azathioprine, cyclosporin), etc. Groups with anti-inflammatory activity, such as sulindac, etodolac, ketoprofen and ketorolac, are also of use. Other drugs of use in conjunction with the present invention will be apparent to those of skill in the art.

When the affinity moiety is a chelating agent, crown ether or cyclodextrin, host-guest chemistry will dominate the interaction between the affinity moiety and the target. The use of host-guest chemistry allows a great degree of affinity-moiety-target specificity to be engineered into a device of the invention. The use of these compounds to bind to specific compounds is well known to those of skill in the art. See, for example, Pitt et al. “The Design of Chelating Agents for the Treatment of Iron Overload,” In, INORGANIC CHEMISTRY IN BIOLOGY AND MEDICINE; Martell, A. E., Ed.; American Chemical Society, Washington, D.C., 1980, pp. 279-312; Lindoy, L. F., THE CHEMISTRY OF MACROCYCLIC LIGAND COMPLEXES; Cambridge University Press, Cambridge,1989; Dugas, H., BIOORGANIC CHEMISTRY; Springer-Verlag, New York, 1989, and references contained therein.

Additionally, a number of routes allowing the attachment of chelating agents, crown ethers and cyclodextrins to other molecules is available to those of skill in the art. See, for example, Meares et al., “Properties of In vivo Chelate-Tagged Proteins and Polypeptides.” In, MODIFICATION OF PROTEINS: FOOD, NUTRITIONAL, AND PHARMACOLOGICAL ASPECTS;”Feeney, R. E., Whitaker, J. R., Eds., American Chemical Society, Washington, D.C., 1982, pp. 370-387; Kasina et al. Bioconjugate Chem. 9:108-117 (1998); Song et al, Bioconjugate Chem. 8:249-255 (1997).

In an exemplary embodiment, the affinity moiety is a polyaminocarboxylate chelating agent such as ethylenediaminetetraacetic acid (EDTA) or diethylenetriaminepentaacetic acid (DTPA), which is attached to an amine on the substrate, or spacer arm, by utilizing the commercially available dianhydride (Aldrich Chemical Co., Milwaukee, Wis.). When complexed with a metal ion, the metal chelate binds to tagged species, such as polyhistidyl-tagged proteins, which can be used to recognize and bind target species. Alternatively, the metal ion itself, or a species complexing the metal ion can be the target.

In further exemplary embodiment, the affinity moiety forms an inclusion complex with the target of interest. In a preferred embodiment, the affinity moiety is a cyclodextrin or modified cyclodextrin. Cyclodextrins are a group of cyclic oligosaccharides produced by numerous microorganisms. Cyclodextrins have a ring structure which has a basket-like shape. This shape allows cyclodextrins to include many kinds of molecules into their internal cavity. See, for example, Szejtli, J., CYCLODEXTRINS AND THEIR INCLUSION COMPLEXES; Akademiai Klado, Budapest, 1982; and Bender et al., CYCLODEXTRIN CHEMISTRY, Springer-Verlag, Berlin, 1978. Cyclodextrins are able to form inclusion complexes with an array of organic molecules including, for example, drugs, pesticides, herbicides and agents of war. See, Tenjarla et al., J. Pharm. Sci. 87:425-429 (1998); Zughul et al., Pharm. Dev. Technol. 3:43-53 (1998); and Albers et al., Crit. Rev. Ther. Drug Carrier Syst. 12:311-337 (1995). Importantly, cyclodextrins are able to discriminate between enantiomers of compounds in their inclusion complexes. Thus, in one preferred embodiment, the invention provides for the detection of a particular enantiomer in a mixture of enantiomers. See, Koppenhoefer et al. J. Chromatogr. A 793:153-164 (1998). The cyclodextrin affinity moiety can be attached to a spacer arm or directly to the substrate. See, Yamamoto et al., J. Phys. Chem. B 101:6855-6860 (1997). Methods to attach cyclodextrins to other molecules are well known to those of skill in the chromatographic and pharmaceutical arts. See, Sreenivasan, K. J. Appl. Polym. Sci. 60:2245-2249 (1996).

In a further preferred embodiment, the affinity moiety is selected from nucleic acid species such as aptamers and aptazymes that recognize specific targets. Aptamers are nucleic acid-based binding-receptors (analogous to antibodies) that are engineered and screened for specific binding properties. Aptamers have been selected against a surprising range of analytes, from ions to peptides to supramolecular structures. Aptamers have even been selected against whole organisms (Xu et al., 1996; Weiss et al., 1997; Convery et al., 1998; Famulok, 1999; Homann and Hu, 1999).

The biophysical characteristics of aptamers make them extremely competitive with antibodies. Aptamers typically bind proteins with K_{d}s in the nanomolar range (Gold et al., 1995), and can distinguish between analytes that differ by as little as a single methyl group (Ellington, 1994. Similarly, aptamers can discriminate between proteins that differ by only a few amino acids (Conrad et al., 1994; Eaton et al., 1995; Hirao et al., 1999).

Aptamer chemistry can be controlled by introducing modified nucleotides. For instance, modified RNA aptamers are extraordinarily stable, even in nuclease-rich environments, such as sera or urine (Green et al., 1995). More importantly, modifications can also be introduced to reduce NSB.

Both antibody and aptamer receptors are fully compatible with each other, and offer the potential for exquisitely high affinity binding. They can each recognize either the same or different epitopes in a protein or cell surface, and mixtures of antibodies and aptamers can even be used in sandwich assays.

To produce aptamers of use in the present invention, functional nucleic acids are selected from random sequence pools that span from 30 to 200 random sequence positions and contain more than 10¹⁵ members. Affinity chromatography is used to separate active sequences from the population, which are amplified by reverse transcription, PCR amplification, or in vitro transcription. Multiple selection/amplification rounds isolate those few binding or catalytic species with the highest affinities and specificities for the analyte molecule. These methods are well established, and yield aptamers that have K_{d}s in the sub-nanomolar range and aptazymes with activation ratios as high as 75,000. The chemistries of oligonucleotide pools (RNA, DNA, or modified RNA), pool lengths, and selection stringencies can be systematically varied to identify the best possible receptors.

Aptazymes are nucleic acids that can catalyze reactions and act as enzymes. Aptazymes are allosteric ribozymes that are activated in the presence of an effector molecule (either chemical or biological), and transduce a non-covalent recognition event into the production of a new covalent bond via ligation. Aptazymes have been developed that are activated over 1,600-fold by a small molecule such as theophylline (Robertson et al., Nucleic Acids Research 28:1751-1759 (2000)), 10,000-fold by an oligonucleotide (Robertson et al., Nature Biotechnol 17:62-66 (1999)), and 75,000-fold by a protein (tyrosyl tRNA synthetase). The allosteric activation parameters of aptazymes used in the present invention are preferably 2-3 orders of magnitude greater than those typically observed for allosteric proteins.

When the affinity moiety is used to detect an organism, it is preferred to use as an affinity moiety antigens common to a species, key virulence determinants, adhesins, and the like. For example, identifying gram-negative bacterial pathogens can rely on an affinity moiety that binds to a selected conserved surface protein, structures related to a type III secretion system, TolC-like molecules involved in macromolecular transport including multi-drug resistance, flagellae, pilli, certain toxins, etc (Koronakis et al., Nature 405:914-920 (2000)).

For each cell marker, it is preferred to use an affinity moiety that recognizes surface epitopes conserved in different serotypes or among phylogenetically related organisms. For example, to identify Salmonella typhimurium, affinity moieties for conserved antigens such as OmpC (a porin which show a high degree of conservation of certain surface epitopes (Singh et al., Infect. Immun. 63:4600-5 (1995))), SpiA (the YscC homologue of Salmonella, a protein critical for the function of Type III secretion systems (Hueck C. J., Microbiol Mol. Biol. Rev 62:379-433 (1998))), TolC (a key protein in extracellular transport (Koronakis et al., Nature 405:914-920 (2000))), OmpT (a virulence factor), PpdD (a type VI pilin), EspA (the “syringe” in type III secretion in enteropathogenic E.coli) and FimA (the major protein of type I pili). can be used. YscC and other Type III secretion components are particularly preferred for diagnostic purposes as affinity moieties binding to the conserved C-terminal region can be used to confirm the presence of protein export machinery while simultaneously using affinity moieties for the N-terminal region for species identification.

A similar approach can be employed for any other bacterium of interest. For example, markers for Gram-positive bacteria are also known, such as conserved flagellar genes and the highly conserved sortase (critical for surface protein localization). (Mazmanian et al., Science 285:760-3 (2000)) (Hueck C. J., Microbiol Mol. Biol. Rev 62:379-433 (1998)) In another exemplary embodiment, the affinity moiety interacts with an organism-derived molecular target, which is preferably abundant at an early stage of infection (e.g., an exotoxin). Representative toxin subunits include, but are not limited to, the protective antigen to B. anthracis toxin (PABat) and the ricin toxin subunit B (RtsB). In a preferred embodiment, the affinity moiety is an antibody against the toxin. Yet another preferred affinity moiety is specific for verotoxin.

G. Targets

The methods of the present invention can be used to detect any target, or class of targets, which interact with an affinity moiety in a detectable manner. The interaction between the target and affinity moiety can be any physicochemical interaction, including covalent bonding, ionic bonding, hydrogen bonding, van der Waals interactions, attractive electronic interactions and hydrophobic/hydrophilic interactions.

In an exemplary embodiment, the interaction is an ionic interaction. In this embodiment, an acid, base, metal ion or metal ion-binding ligand is the target. In a further exemplary embodiment, the interaction is a hydrogen bonding interaction. In a preferred embodiment, the hybridization of an immobilized nucleic acid to a nucleic acid having a complementary sequence is detected. In another preferred embodiment, the interaction is between an enzyme or receptor and a small molecule which binds thereto. One of skill in the art will appreciate that an affinity moiety in one assay, can be a target in another assay. The terms “target” and “affinity moiety” are not absolute, but are dependent on what is being detected (“target”) by interaction with an affinity moiety.

The target can be labeled with a quantum dot either directly or indirectly through interacting with a second species to which a quantum dot is bound. When a second labeled species is used as an indirect labeling agent, it is selected from any species that is known to interact with the target species. Preferred second labeled species include, but are not limited to, antibodies, aptazymes, aptamers, streptavidin, and biotin.

The target can be labeled either before or after it interacts with the affinity moiety. The target molecule can be labeled with a single quantum dot or more than one quantum dot. Where the target species is multiply labeled with more than one quantum dot, the individual quantum dots are preferably distinguishable from each other. Properties on the basis of which the individual quantum dots can be distinguished include, but are not limited to, fluorescence wavelength, absorption wavelength, fluorescence emission, fluorescence excitation spectrum, ultraviolet light absorbance, visible light absorbance, fluorescence quantum yield, fluorescence lifetime, light scattering and combinations thereof. In a preferred embodiment, the multiple quantum dots are visually distinguishable as two or more colors. In another preferred embodiment, the colors of the two or more quantum dots combine to produce a color, which is different from either of the colors from which it is derived.

In presently preferred embodiments, the target is a member selected from the group consisting of acids, bases, organic ions, inorganic ions, pharmaceuticals, herbicides, pesticides, chemical warfare agents, organisms, noxious gases and biomolecules. Each of these targets can be detected as a vapor or a liquid. These targets can be present as components in mixtures of structurally unrelated compounds, racemic mixtures of stereoisomers, non-racemic mixtures of stereoisomers, mixtures of diastereomers, mixtures of positional isomers or as pure compounds. Within the scope of the invention is method to detect a particular target of interest without interference from other substances within a mixture.

Organic ions, which are substantially non-acidic and non-basic (e.g., quaternary alkylammonium salts) can be detected by an affinity moiety. For example, an affinity moiety with ion exchange properties is useful in the present invention. A specific example is the exchange of a cation such as dodecyltrimethylammonium cation for a metal ion such as sodium, using a spacer arm presenting a negatively charged species. Affinity moieties that form inclusion complexes with organic cations are also of use. For example, crown ethers and cryptands can be used to form inclusion complexes with organic ions such as quaternary ammonium cations.

Inorganic ions such as metal ions and complex ions (e.g., SO₄⁻², PO₄⁻³) can also be detected using the device and method of the invention. Metal ions can be detected, for example, by their complexation or chelation by agents bound to a spacer arm or the substrate. In this embodiment, the affinity moiety can be a simple complexing moiety (e.g., carboxylate, amine, thiol) or can be a more structurally complicated agent (e.g., ethylenediaminepentaacetic acid, crown ethers, aza crowns, thia crowns).

Complex inorganic ions can be detected by, for example, their ability to compete with ligands for bound metal ions in ligand-metal complexes. When a ligand bound to a spacer arm or a substrate forms a metal-complex having a thermodynamic stability constant, which is less than that of the complex between the metal and the complex ion, the complex ion will replace the metal ion on the immobilized ligand. Methods of determining stability constants for compounds formed between metal ions and ligands are well known to those of skill in the art. Using these stability constants, substrates including affinity moieties that are specific for particular ions can be manufactured. See, Martell, A.E., Motekaitis, R. J., DETERMINATION AND USE OF STABILITY CONSTANTS, 2d Ed., VCH Publishers, New York 1992.

Small molecules such as pesticides, herbicides, agents of war, and the like can be detected by the use of a number of different affinity moiety motifs. Acidic or basic components can be detected as described above. A target's metal binding capability can also be used to advantage, as described above for complex ions. Additionally, if these targets bind to an identified biological structure (e.g., a receptor), the receptor can be immobilized on the substrate, a spacer arm. Techniques are also available in the art for raising antibodies which are highly specific for a particular species. Thus, it is within the scope of the present invention to make use of antibodies against small molecules, pesticides, agents of war and the like for detection of those species. Techniques for raising antibodies to herbicides, pesticides and agents of war are known to those of skill in the art. See, Harlow, Lane, MONOCLONAL ANTIBODIES: A LABORATORY MANUAL, Cold Springs Harbor Laboratory, Long Island, N. Y., 1988.

In another exemplary embodiment, the target is detected by binding to an immobilized affinity moiety is an organophosphorous compound such as an insecticide or an agent of war (e.g., VX, O-ethyl-S-(2-diisopropylaminoethyl)-methylthiophosphonate). Exemplary compounds which exhibit affinity for organophosphorous agents include, but are not limited to, Cu⁺²-diamine, triethylentetraamine-Cu⁺²-chloride, tetraethylenediamine-Cu⁺²-chloride and 2,2′-bipyridine-Cu⁺²-chloride. See, U.S. Pat. No. 4,549,427, issued to Kolesar, on Oct. 29, 1985.

In a preferred embodiment, the herbicides are preferably members of the group consisting of triazines, haloacetanilides, carbamates, toluidines, ureas, plant growth hormones and diphenyl ethers. Included within these broad generic groupings are commercially available herbicides such as phenoxyl alkanoic acids, bipyridiniums, benzonitriles, dinitroanilines, acid amides, carbamates, thiocarbamates, heterocyclic nitrogen compounds including triazines, pyridines, pyridazinones, sulfonylureas, imidazoles, substituted ureas, halogenated aliphatic carboxylic acids, inorganics, organometallics and derivatives of biologically important amino acids.

Pesticides preferred for detection using the present invention include bactericides (e.g., formaldehyde), fumigants (e.g., bromomethane), fungicides (e.g., 2-phenylphenol, biphenyl, mercuric oxide, imazalil), acaricides (e.g., abamectin, bifenthrin), insecticides (e.g., imidacloprid, prallethrin, cyphenothrin)

In the embodiments discussed above, the preferred agent of war is a member of the group consisting of mustard and related vesicants including the agents known as HD, Q, T, HN1, HN2, HN3, nerve agents, particularly the organic esters of substituted phosphoric acid including tabun, sarin, isopropyl methylphosphonofluoridate, soman pinacolyl methylphosphonofluoridate. Other detectable targets include incapacitants such as BZ, 3-quinuclidinyl benzilate and irritants such as the riot control compound CS. Other agents of war include infectious organisms such as anthrax, E. coli, and the like. Within the scope of the present invention is the detection and/or quantification of any infectious organism.

The present invention also provides a device and a method for detecting noxious gases such as CO, CO₂, SO₃, H₂SO₄, SO₂, NO, NO₂, N₂O₄ and the like. In a preferred embodiment, the substrate or a spacer arm includes at least one compound capable of detecting the gas. Useful compounds include, but are not limited to, palladium compounds selected from the group consisting of palladium sulfate, palladium sulfite, palladium pyrosulfite, palladium chloride, palladium bromide, palladium iodide, palladium perchlorate, palladium complexes with organic complexing reagents and mixtures thereof. Other compounds of use in practicing this embodiment of the present invention include, molybdenum compounds such as silicomolybdic acid, salts of silicomolybdic acid, molybdenum trioxide, heteropolyacids of molybdenum containing vanadium, copper or tungsten, ammonium molybdate, alkali metal or alkaline earth salts of molybdate anion, heteropolymolybdates and mixtures thereof.

Still further useful gas detecting compounds include, copper salts and copper complexes with an available coordination site. Alpha-cyclodextrin, beta-cyclodextrin, modified alpha- and beta-cyclodextrins, gamma-cyclodextrin and mixtures thereof are of use in practicing the present invention. See, U.S. Pat. No. 5,618,493, issued to Goldstein et al. on Apr. 8, 1997 and No. 5,071,526, issued to Pletcher et al. on Dec. 10, 1991.

In another preferred gas detecting embodiment, the substrate, or a spacer arm is derivatized with a compound selected from the group consisting of amorphous hemoglobin, crystalline hemoglobin, amorphous heme, crystalline heme and mixtures thereof. The heme serves as an affinity moiety which is reactive towards the gas. See, U.S. Pat. No. 3,693,327, issued to Scheinberg, on Sep. 26, 1972.

H. Assays

The method of the present invention is useful in performing assays of substantially any format including, but not limited to immunoassays, competitive assays, nucleic acid binding assays, sandwich assays and the like. The following discussion focuses on the use of the methods of the invention in practicing immunoassays. This focus is for clarity of illustration only and is not intended to define or limit the scope of the invention. Those of skill in the art will appreciate that the method of the invention is broadly applicable to any assay technique for detecting the presence and/or amount of a target in which the immobilization of fluorescence on a surface has a quantitative relation to the amount of target present.

Assays based on specific binding reactions have been used for detecting a wide variety of targets such as nucleic acids, drugs, hormones, enzymes, proteins, antibodies, and infectious agents in various biological fluids and tissue samples. In general, the assays consist of a target, a binding moiety specific for the target, and a detectable label. Immunological assays involve reactions between immunoglobulins (antibodies) which are capable of binding with specific antigenic determinants of various compounds and materials (antigens). Other types of reactions include binding between complementary strands of DNA, RNA or the like, avidin and biotin, protein A and immunoglobulins, lectins and sugar moieties and the like. See, for example, U.S. Pat. No. 4,313,734, issued to Leuvering; U.S. Pat. No. 4,435,504, issued to Zuk; U.S. Pat. Nos. 4,452,901 and 4,960,691, issued to Gordon; and U.S. Pat. No. 3,893,808, issued to Campbell.

The present invention provides assays that are useful for confirming the presence or absence of a target in a sample and for quantitating a target in a sample. Exemplary assay formats with which the invention can be used include, but are not limited to competitive assays, and sandwich assays. The invention is further illustrated using these two assay formats. The focus of the following discussion on competitive assays and sandwich assays is for clarity of illustration and is not intended to either define or limit the scope of the invention. Those of skill in the art will appreciate that the invention described herein can be practiced in conjunction with a number of other assay formats. An exemplary assay format is set forth in FIG. 10.

In an exemplary competitive binding assay, quantum dot-labeled reagents and unlabeled target compounds compete for binding sites on an affinity moiety. After an incubation period, unbound materials are optionally washed off and the amount of labeled reagent bound to the site is compared to reference amounts for determination of the target concentration in the assay mixture. Other competitive assay motifs using labeled target and/or labeled affinity moiety and/or labeled reagents will be apparent to those of skill in the art.

A second type of assay is known as a sandwich assay and generally involves contacting an assay mixture with a surface having immobilized thereon a first affinity moiety specific for a target. A second solution comprising a labeled binding material is then added to the assay. The labeled binding material will bind to any target that is bound to the affinity moiety. The assay system is then subjected to an optional wash step to remove labeled binding material that failed to bind with the target and the amount of labeled material remaining is ordinarily proportional to the amount of bound target. In representative assays one or more of the target, affinity moiety or binding material is labeled with a quantum dot.

In addition to detecting an interaction between an affinity moiety and a target, it is frequently desired to quantitate the magnitude of the affinity between two or more binding partners. The format of an assay for extracting affinity data for two molecules can be understood by reference to an exemplary embodiment in which a ligand that is known to bind to a receptor is displaced by an antagonist to that receptor. Other variations on this format will be apparent to those of skill in the art. The competitive format is well known to those of skill in the art. See, for example, U.S. Pat. Nos. 3,654,090 and 3,850,752.

The binding of an antagonist to a receptor can be assayed by a competitive binding method using a ligand for that receptor and the antagonist. One of the three binding partners (i.e., the ligand, antagonist or receptor) is bound to the substrate. In an exemplary embodiment, the receptor is bound to the substrate. Various concentrations of unlabeled ligand can be added to different substrate regions. A labeled antagonist is then applied to each region to a chosen final concentration. The mixtures will generally be incubated at room temperature for a preselected time. The receptor-bound labeled antagonist can be separated from the unbound labeled antagonist by filtration, washing or a combination of these techniques. Bound label remaining on the substrate can be measured as discussed above. A number of variations on this general experimental procedure will be apparent to those of skill in the art.

Competition binding data can be analyzed by a number of techniques, including nonlinear least-squares curve fitting procedure. When the ligand is an antagonist for the receptor, this method provides the IC50 of the antagonist (concentration of the antagonist which inhibits specific binding of the ligand by 50% at equilibrium). The IC50 is related to the equilibrium dissociation constant (Ki) of the antagonist based on the Cheng and Prusoff equation: Ki=IC50/(1+L/Kd), where L is the concentration of the ligand used in the competitive binding assay, and Kd is the dissociation constant of the ligand as determined by Scatchard analysis. These assays are described, among other places, in Maddox et al., J. Exp Med., 158: 1211 (1983); Hampton et al., SEROLOGICAL METHODS, A LABORATORY MANUAL, APS Press, St. Paul, Minn., 1990.

The method of the present invention is also of use in screening libraries of compounds, such as combinatorial libraries. The synthesis and screening of chemical libraries to identify compounds, which have novel pharmacological and material science properties is now a common practice. Libraries that have been synthesized include, for example, collections of oligonucleotides, oligopeptides, and small and large molecular weight organic or inorganic molecules. See, Moran et al., PCT Publication WO 97/35198, published Sep. 25, 1997; Baindur et al., PCT Publication WO 96/40732, published Dec. 19, 1996; Gallop et al., J. Med. Chem. 37:1233-51 (1994).

Virtually any type of compound library can be probed using the method of the invention, including peptides, nucleic acids, saccharides, small and large molecular weight organic and inorganic compounds. In a presently preferred embodiment, the libraries synthesized comprise more than 10 unique compounds, preferably more than 100 unique compounds and more preferably more than 1000 unique compounds.

The nature of these libraries is better understood by reference to peptide-based combinatorial libraries as an example. The present invention is useful for assembling peptide-based combinatorial libraries, but it is not limited to these libraries. The methods of the invention can be used to screen libraries of essentially any molecular format, including small organic molecules, carbohydrates, nucleic acids, polymers, organometallic compounds and the like. Thus, the following discussion, while focusing on peptide libraries, is intended to be illustrative and not limiting.

Libraries of peptides and certain types of peptide mimetics, called “peptoids”, are assembled and screened for a desirable biological activity by a range of methodologies (see, Gordon et al., J. Med Chem., 37: 1385-1401 (1994); Geysen, (Bioorg. Med. Chem. Letters, 3: 397-404 (1993); Proc. Natl. Acad Sci. USA, 81: 3998 (1984); Houghton, Proc. Natl. Acad. Sci. USA, 82: 5131 (1985); Eichler et al., Biochemistry, 32: 11035-11041 (1993); and U.S. Pat. No. 4,631,211); Fodor et al., Science, 251: 767 (1991); Huebner et al. (U.S. Pat. No. 5,182,366). Small organic molecules have also been prepared by combinatorial means. See, for example, Camps. et al., Annaks de Quimica, 70: 848 (1990); U.S. Pat. No. 5,288,514; U.S. Pat. No. 5,324,483; Chen et al., J. Am. Chem. Soc., 116: 2661-2662 (1994).

In an exemplary embodiment, the library to be screened includes compounds that target a particular enzyme. The compound library is immobilized to a substrate and the library is probed with a derivative of the enzyme labeled with a quantum dot. Other methods for using the methods of the invention to screen combinatorial libraries will be apparent to those of skill in the art.

Additionally, a binding domain of a receptor, for example, can serve as the focal point for a drug discovery assay, where, for example, the receptor is immobilized, and incubated both with agents (i.e., ligands) known to interact with the binding domain thereof, and a quantity of a particular drug or inhibitory agent under test. The extent to which the drug binds with the receptor and thereby inhibits receptor-ligand complex formation can then be measured. Such possibilities for drug discovery assays are contemplated herein and are considered within the scope of the present invention. Other focal points and appropriate assay formats will be apparent to those of skill in the art.

I. Informatics

As high-resolution, high-sensitivity datasets acquired using the methods of the invention become available to the art, significant progress in the areas of diagnostics, therapeutics, drug development, biosensor development, and other related areas will occur. For example, disease markers can be identified and utilized for better confirmation of a disease condition or stage (see, U.S. Pat. Nos. 5,672,480; 5,599,677; 5,939,533; and 5,710,007). Subcellular toxicological information can be generated to better direct drug structure and activity correlation (see, Anderson, L., “Pharmaceutical Proteomics: Targets, Mechanism, and Function,” paper presented at the IBC Proteomics conference, Coronado, Calif. (Jun. 11-12, 1998)). Subcellular toxicological information can also be utilized in a biological sensor device to predict the likely toxicological effect of chemical exposures and likely tolerable exposure thresholds (see, U.S. Pat. No. 5,811,231). Similar advantages accrue from datasets relevant to other biomolecules and bioactive agents (e.g., nucleic acids, saccharides, lipids, drugs, and the like).

Thus, in another preferred embodiment, the present invention provides a database that includes at least one set of data assay data. The data contained in the database is acquired using a method of the invention and/or a quantum dot-labeled species of the invention either singly or in a library format. The database can be in substantially any form in which data can be maintained and transmitted, but is preferably an electronic database. The electronic database of the invention can be maintained on any electronic device allowing for the storage of and access to the database, such as a personal computer, but is preferably distributed on a wide area network, such as the World Wide Web.

The focus of the present section on databases, which include peptide sequence specificity data is for clarity of illustration only. It will be apparent to those of skill in the art that similar databases can be assembled for any assay data acquired using an assay of the invention.

The compositions and methods described herein for identifying and/or quantitating the relative and/or absolute abundance of a variety of molecular and macromolecular species from a biological sample provide an abundance of information, which can be correlated with pathological conditions, predisposition to disease, drug testing, therapeutic monitoring, gene-disease causal linkages, identification of correlates of immunity and physiological status, among others. Although the data generated from the assays of the invention is suited for manual review and analysis, in a preferred embodiment, prior data processing using high-speed computers is utilized.

An array of methods for indexing and retrieving biomolecular information is known in the art. For example, U.S. Pat. Nos. 6,023,659 and 5,966,712 disclose a relational database system for storing biomolecular sequence information in a manner that allows sequences to be catalogued and searched according to one or more protein function hierarchies. U.S. Pat. No. 5,953,727 discloses a relational database having sequence records containing information in a format that allows a collection of partial-length DNA sequences to be catalogued and searched according to association with one or more sequencing projects for obtaining full-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which can be viewed as a tree structure or as the merger of two or more such tree structures.

The present invention provides a computer database comprising a computer and software for storing in computer-retrievable form assay data records cross-tabulated, for example, with data specifying the source of the target-containing sample from which each sequence specificity record was obtained.

In an exemplary embodiment, at least one of the sources of target-containing sample is from a tissue sample known to be free of pathological disorders. In a variation, at least one of the sources is a known pathological tissue specimen, for example, a neoplastic lesion or a tissue specimen containing a pathogen such as a virus, bacteria or the like. In another variation, the assay records cross-tabulate one or more of the following parameters for each target species in a sample: (1) a unique identification code, which can include, for example, a target molecular structure and/or characteristic separation coordinate (e.g., electrophoretic coordinates); (2) sample source; and (3) absolute and/or relative quantity of the target species present in the sample.

The invention also provides for the storage and retrieval of a collection of target data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the target data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor). In one embodiment, the invention provides such storage devices, and computer systems built therewith, comprising a bit pattern encoding a protein expression fingerprint record comprising unique identifiers for at least 10 target data records cross-tabulated with target source.

When the target is a peptide or nucleic acid, the invention preferably provides a method for identifying related peptide or nucleic acid sequences, comprising performing a computerized comparison between a peptide or nucleic acid sequence assay record stored in or retrieved from a computer storage device or database and at least one other sequence. The comparison can include a sequence analysis or comparison algorithm or computer program -nembodiment thereof (e.g., FASTA, TFASTA, GAP, BESTFIT) and/or the comparison may be of the relative amount of a peptide or nucleic acid sequence in a pool of sequences determined from a polypeptide or nucleic acid sample of a specimen.

The invention also preferably provides a magnetic disk, such as an IBM-compatible (DOS, Windows, Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or 10BaseT), telephone line, ISDN line, wireless network, optical fiber, or other suitable signal tramnission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like, wherein the signal includes (in native or encrypted format) a bit pattern encoding data from an assay or a database comprising a plurality of assay results obtained by the method of the invention.

In a preferred embodiment, the invention provides a computer system for comparing a query target to a database containing an array of data structures, such as an assay result obtained by the method of the invention, and ranking database targets based on the degree of identity and gap weight to the target data. A central processor is preferably initialized to load and execute the computer program for alignment and/or comparison of the assay results. Data for a query target is entered into the central processor via an I/O device. Execution of the computer program results in the central processor retrieving the assay data from the data file, which comprises a binary description of an assay result.

The target data or record and the computer program can be transferred to secondary memory, which is typically random access memory (e.g., DRAM, SRAM, SGRAM, or SDRAM). Targets are ranked according to the degree of correspondence between a selected assay characteristic (e.g., binding to a selected affinity moiety) and the same characteristic of the query target and results are output via an I/O device. For example, a central processor can be a conventional computer (e.g., Intel Pentium, PowerPC, Alpha, PA-8000, SPARC, MIPS 4400, MIPS 10000, VAX, etc.); a program can be a commercial or public domain molecular biology software package (e.g., UWGCG Sequence Analysis Software, Darwin); a data file can be an optical or magnetic disk, a data server, a memory device (e.g., DRAM, SRAM, SGRAM, SDRAM, EPROM, bubble memory, flash memory, etc.); an I/O device can be a terminal comprising a video display and a keyboard, a modem, an ISDN terminal adapter, an Ethernet port, a punched card reader, a magnetic strip reader, or other suitable I/O device.

The invention also preferably provides the use of a computer system, such as that described above, which comprises: (1) a computer; (2) a stored bit pattern encoding a collection of peptide sequence specificity records obtained by the methods of the invention, which may be stored in the computer; (3) a comparison target, such as a query target; and (4) a program for alignment and comparison, typically with rank-ordering of comparison results on the basis of computed similarity values.

J. Haplotyping

The single target counting methods and assays described above may be adapted for use in detecting and quantitating target nucleic acid sequences or species in a sample (e.g., DNA, RNA, etc.). As such, in one aspect, the present invention a method of detecting the presence of at least one target nucleic acid sequence in a sample, said method comprising: labeling at least one target nucleic acid sequence with at least one quantum dot; and detecting the labeled target nucleic acid sequence by detecting fluorescence emitted by the at least one quantum dot, wherein the detection of fluorescence in the sample indicates the presence of at least one target nucleic acid sequence. Preferably, the method further comprises quantitating the target nucleic acid sequence by analyzing the detected emitted fluorescence. In one preferred embodiment, the method further comprises transcribing the target nucleic acid sequence. In another preferred embodiment, the target nucleic acid sequence is DNA and transcribing comprises using a primer which anneals to a conserved region of the DNA and transcribes a polymorphic region of the DNA when extended.

In another embodiment, the present invention provides a method of detecting the presence of a target nucleic acid sequence in a sample, the method comprising: transcribing a target nucleic acid sequence using a primer that is complementary to a portion of said target nucleic acid sequence and that comprises an immobilizable label to form an immobilizable target nucleic acid sequence; immobilizing the immobilizable target nucleic acid sequence on a solid support to form an immobilized target nucleic acid sequence; probing the immobilized target nucleic acid sequence using a sequence-tagged hybridization probe, wherein the sequence-tagged hybridization probe is complementary to a portion of the target nucleic acid squence; labeling the immobilized target sequence with a quantum dot conjugate, wherein the quantum dot conjugate comprises a quantum dot and a nucleic acid sequence that is complementary to a portion of the sequence-tagged hybridization probe; and detecting the labeled immobilized target nucleic acid sequence by detecting fluorescence emitted by the quantum dot, wherein the detection of fluorescence in the sample indicates the presence of the target nucleic acid sequence.

In a further embodiment, the present invention provides a method of selecting a mutant DNA away from a wild type DNA, said method comprising providing mutant DNA and wild type DNA target nucleic acid sequence having attached sequence-tagged hybridization probes; adding at least a first quantum dot having a conjugated oligonucleotide tag which is complementary to sequence tags on mutant DNA; adding at least a second quantum dot having a conjugated oligonucleotide tag which is complementary to sequence tags on wild type DNA; and detecting the quantum dots and correlating the quantum dots to either the mutant DNA or wild type DNA.

For the sake of example, K-ras will be used as the prototypical target in plasma. The target DNA originates from necrosed tumor cells. Kirsten (K)-ras oncogene mutations are present in up to 95% of pancreatic and bile duct adenocarcinomas, with codon 12 K-ras mutations accounting for approximately 75% of this total. The presence of a mutation is nearly 100% specific for carcinoma. Codon 12 K-ras assays are currently used for the tissue diagnosis of pancreas and bile duct carcinoma.

Several different qualitative assays for detecting the presence of K-ras DNA circulating in human plasma have been reported in the literature. These methods generally involve PCR amplification of target, and include technologies such as, sequence specific hybridization, sequence specific PCR, SSCP, or TaqMan, RFLP or DNA sequencing. None of these methods report focused attempts at quantitating the level of K-ras target in the plasma. In general, assays involving target amplification are not desirable for quantitative assays, since the efficiency of the amplification step can vary dramatically between different samples. The assays of the present invention involves directly detecting and counting the target DNA from the patient sample thus measuring the amount of K-ras DNA in the sample without an amplification step. This is accomplished with the use of individual or single molecules of quantum dots to detect each target molecule.

An in vitro assay has been designed accordingly. Specifically, the assay is designed to detect and quantitate a target species, in this case K-ras, in plasma. The assay has sufficient sensitivity to achieve this with very low levels of K-ras target. In addition, the assay is used to determine the genotype for 5-6 point mutations located in the K-ras DNA and to selected mutant DNA away from the wild type. Typically, the wild type is the predominant sequence within the sample.

In this exemplar assay, synthetic or plasmid target containing 2-3 K-ras mutations (e.g. exon 12) and synthetic or plasmid target of wild type K-ras are used as the target species. Sequence information for the entire target and mutations should be known. A primer is designed which anneals to a conserved region, and when extended will transcribe the polymorphic region of K-ras. In this embodiment, 5′ biotinylated oligonucleotide primers are used for simultaneously labeling and transcribing the target sequence. It will be readily apparent to those of skill in the art that the primer can be labeled with any immobilizable label or tag. A number of different immobilizable tags and capture moieties can be used that are based upon numerous molecular interactions well described in the literature. For instance, where an immobilizable tag has a natural binder (e.g., biotin, protein A or protein G), it can be used in conjunction with an appropriate capture moiety (e.g., avidin, streptavidin, neutravidin, the Fe region of an immunoglobulin, etc.). Moreover, antibodies to molecules having natural capture moieties, such as biotin, are also widely available as appropriate capture moiety or tag binders (see, SIGMA Immunochemicals 1998 catalogue, SIGMA Chemical Co. (St. Louis Mo.)). Conditions for heat denaturing the sample, allowing the primer to anneal and the reagents/conditions for primer extension (DNA polymerase, dNTPs, etc.) are optimized using standard techniques known to those of skill in the art. FIG. 12 illustrates transcription of the K-ras DNA target, having 3 point mutations, by the biotinylated primer. A spin column method may be used for simultaneously removing unincorporated biotinylated primer, concentrating the sample, and dialyzing the same into hybridization buffer.

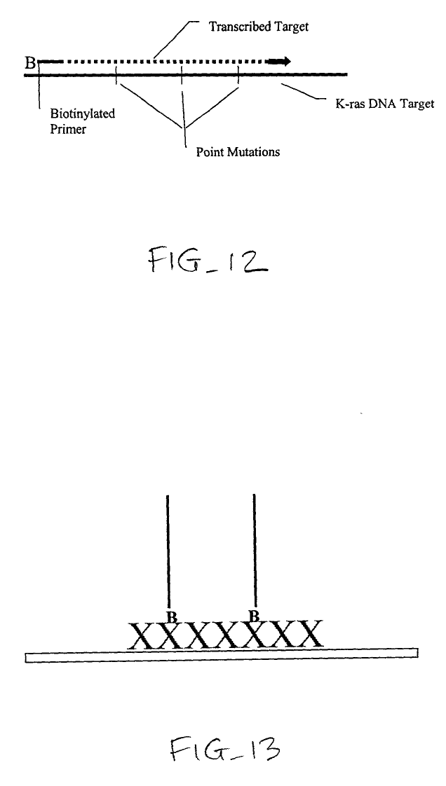

The transcribed, biotinylated target is then captured onto a solid support coated with streptavidin, as shown in FIG. 13. Any solid support or substrate capable of immobilizing the transcribed target nucleic acid can be used. This surface may be glass or plastic in the form of a microscope slide or a 96 microwell plate, for example. There are several commercial sources suitable for this purpose. The primary specifications include low auto-fluorescence, low non-specific binding, compatibility with commercial fluorescent imaging equipment and sufficient affinity for binding the immobilizable target (e.g. the biotinylated target). In any case, unbound strands of sample are then washed away. Optionally, the captured sample strands may be aligned on the surface as shown in FIG. 14.

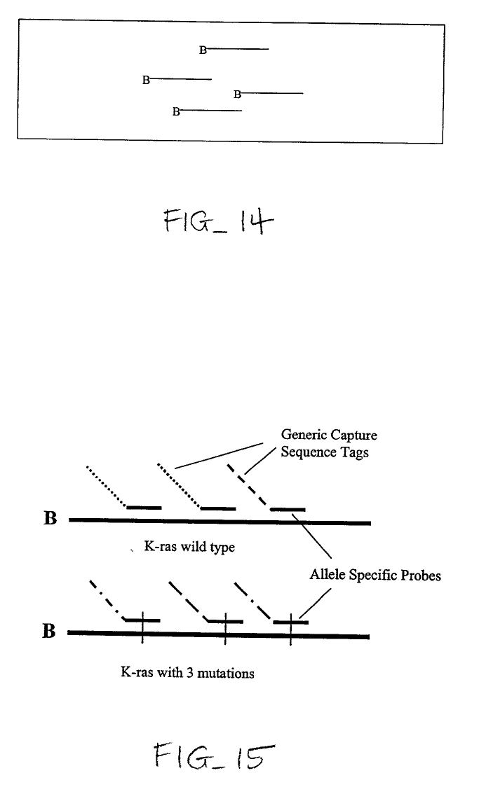

Once immobilized, the target nucleic acid is then detected using a probe that hybridizes to a portion of the target nucleic acid sequence. Specific probes and conditions for hybridizing generic sequence tagged probes to the immobilized target are developed, see U.S. application Ser. No.______ by Lai et al. entitled “Methods and Compositions for Polynucleotide Analysis using Generic Capture Sequences”, filed on Apr. 30, 2001 and incorporated by reference for all purposes. For instance, in the case of K-ras nucleic acid, normal allele-specific oligonucleotide (ASP) probes are tested. Alternatively, the loop probe oligonucleotide, as described in U.S. Provisional Application Ser. No. 60/191,227 filed Mar. 22, 2000 and corresponding U.S. application Ser. No.______ filed Mar. 22, 2001, both of which are incorporated by reference herein for all purposes, may be used. As shown in FIG. 15, the captured strands are probed using sequence-tagged hybridization probes. Conditions are then applied that are sufficiently stringent to remove non-specifically bound probe.

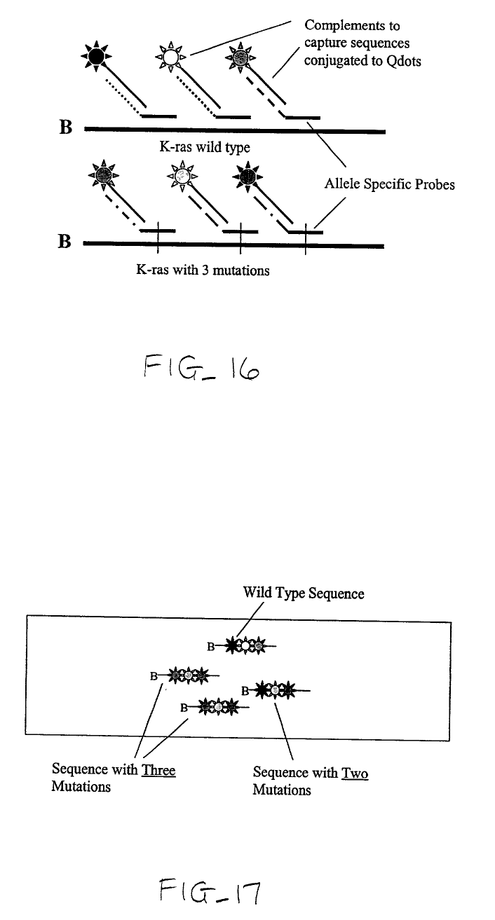

In the next step of the assay, the immobilized target nucleic acid is labeled with at least one quantum dot conjugate, the quantum dot conjugate comprising a quantum dot and a nucleic acid sequence that is complementary, at least in part, to the sequence-tagged hybridization probe. Numerous combinations of quantum dots and nucleic acid sequences can be developed. For instance, six different quantum dot/oligo tags are developed in which the oligos for each color quantum dot are complementary to one of the generic sequence tags used to label the immobilized sample. A cocktail of the six colors of quantum dots are then added so that the quantum dots are bound to the capture sequence tags as shown in FIG. 16. Unbound quantum dots are then removed by washing.

A portion of the surface is then scanned to create an image with sufficient resolution to detect single quantum dots, as illustrated in FIG. 17. The area of the surface which is to be scanned should allow counting of a sufficient number of target molecules and is determined empirically. The target nucleic acid (e.g. mutant target nucleic acid) is identified via the binding and subsequent co-localization of multiple oligo probes, each containing a different color reporter quantum dot. The combination of co-localized colors bound indicates which mutations are present. It will be readily apparent to those of skill in the art that an assay system would ideally include software for automated pattern analysis and identification. Once the patterns for positive/negative signal are determined, already existing image analysis software can be adapted to do automated analysis/counting. The concentration of mutant K-ras/unit volume is a function calculated from the number of co-localized binding events, the volume of sample used, and the ratio of the area scanned to the total area. The number of copies identified within a given scanned area (or multiple areas on the same slide) can be combined with other information, such as the volume of sample tested, the percentage of the total field which is scanned, etc., to calculate the starting concentration of K-ras DNA. In addition, controls may be added into the assay to aid in the quantitation step.

Thus, in summary, the above described assay has five levels of specificity. First is the primer used to extend the labeled target. Sequences not complementary to the primer will not be extended. Second is the capture of the biotinylated target using the streptavidin on the solid phase. DNA not containing biotin label will not be captured. After being washed away, other DNA sequences in the sample cannot interfere in the assay. Third is the probing of the captured K-ras sequences with the allele specific probes. These probes can be developed to have the desired level of specificity. Fourth is the binding of the quantum dot labels to the immobilized probes via the generic capture sequences that were synthesized as part of the allele specific probes. The generic capture sequences can be designed to have very low cross-reactivity. And, fifth is the co-localization of multiple binding events along the same strand of target, allowing non-specific binding events to be identified and discounted. Each of these five factors contributes to reducing false positives in the assay.

The materials, methods and devices of the present invention are further illustrated by the examples that follow. These examples are offered to illustrate, but not to limit the claimed invention.

### EXAMPLES

Example 1 illustrates the concept of single target detection in an exemplary assay. The assay utilizes a glass substrate to which an affinity moiety is passively adsorbed. Single target species bound to the substrate are detected.

Example 2 illustrates a use of single target detection in a exemplary assay for haplotyping DNA samples. The assay utilizes a streptavidin-coated surface to which transcribed, biotinylated target species are captured. Single target species bound to the substrate are detected and quantitated.

### Example 1

To demonstrate the concept of single analyte counting, a dense layer of polyclonal anti-rabbit IgG was passively adsorbed to the surface of standard glass coverslips. Excess antibody was removed and the surfaces were blocked with BSA. Each coverslip was immersed in different concentrations of biotinylated rabbit IgG (10 nM to 100 fM plus PBS control). After binding for 15 minutes, the samples were washed and labeled with streptavidin functionalized quantum dots. After 30 minutes of washing in PBS/1%BSA/0.1% Igepal® at room temperature, samples were imaged with a fluorescence microscope. The points of light in FIG. 3A are signal from single bound analyte molecules, and the density of molecules can be seen decreasing as a function of analyte concentration. The assay was quantified by counting analyte molecules in a defined area. FIG. 3B shows the linearity and sensitivity of this simple assay to densities below 0.001 molecules/μm². This is 100-times more sensitive than the best detection in DNA microarrays using standard fluorophores. The integration time in these images was only 30 ms, suggesting that a small, uncooled CCD could be used for detection. Coupled with the optical system in FIG. 5, this forms the basis of a simple hand-held device.

There are two things to note about FIG. 3. First, this experiment demonstrates not only the feasibility, but also the simplicity of single analyte counting with quantum dots. Assay preparation was at room temperature, with few processing steps. No signal amplification or complicated labeling steps were required and detection was with simple instrumentation and commercially available software. Recent results suggest that the assay, labeling and washing steps can be significantly shortened, allowing the complete assay to be run in under 10 minutes. Second, the absolute sample concentrations used here do not represent the ultimate limit of detection sensitivity for this form of assay. At this level of detection, sensitivity is no longer limited by label detection, but rather the physical performance of the assay in question. The sensitivity of this particular assay was restricted due to the large assay surface-area. At these receptor densities, most of the analyte was removed from solution, reducing the equilibrium binding density and therefore overall sensitivity. In fact, based on theoretical calculations by Ekins (Ekins et al., E Analytica Chimica Acta 227:73 (1989)) (Ekins, R., J. Chem. Ed. 76:769 (1999)), with 100 μm diameter assay spots and antibodies with K_{d}s of only 100 pM, detection of bound ligands at a density comparable to that in FIG. 3 would yield assay sensitivities of less than 1000 molecules/ml of solution. This is an extremely relevant concentration for the early detection of pathogenic infection.

### Example 2

To demonstrate the concept of haplotyping using semiconductor nanocrystals for single analyte counting, K-ras DNA is used as the target species. The K-ras DNA may initially be in buffer and later diluted into normal human plasma. The plasmid target contains 2-3 K-ras mutations and wild type K-ras. 5′ biotinylated oligonucleotide primer or multiple primers are then added to the K-ras DNA target for simultaneous labeling and transcribing as shown in FIG. 12. The unincorporated biotinylated primer is then filtered away from the labeled target. A glass slide or other surface is coated with streptavidin and the biotinylated target captured on the slide, as shown in FIG. 13. Unbound portions of target sample are then washed away. The captured target strands are probed using sequence specific oligonucleotide probes for 2-3 K-ras mutations and wild type K-ras, as shown in FIG. 15. The probes also have attached generic sequence tags. Six different colors of spectrally resolvable quantum dots, each conjugated to a different sequence which was complementary to the generic sequence tags on the probes, are added to the captured, probed target strands as depicted in FIG. 16. Unbound quantum dot/oligo tags are then washed away. The slide is then scanned with a resolution capable of detecting single quantum dots, illustrated in FIG. 17. The mutant target and wild type target are identified via the binding and subsequent co-localization of multiple oligo probes, each containing a different color reporter quantum dot. The target molecules are then counted in the scanned area.

It is understood that the examples and embodiments described herein are for illustrative purposes only and that various modifications or changes in light thereof will be suggested to persons skilled in the art and are to included within the spirit and purview of this application and are considered within the scope of the appended claims. All publications, patents, and patent applications cited herein are hereby incorporated by reference in their entirety for all purposes.

What is claimed is:

1. A method of detecting the presence of at least one target nucleic acid sequence in a sample, said method comprising:
labeling at least one target nucleic acid sequence with at least one quantum dot; and
detecting the labeled target nucleic acid sequence by detecting fluorescence emitted by the at least one quantum dot, wherein the detection of fluorescence in the sample indicates the presence of at least one target nucleic acid sequence.

2. The method as in claim 1, further comprising quantitating the target nucleic acid sequence by analyzing the detected emitted fluorescence.

3. The method as in claim 1, further comprising transcribing the target nucleic acid sequence.

4. The method as in claim 3, wherein the target nucleic acid sequence comprises DNA and transcribing comprises using a primer which anneals to a conserved region of the DNA and transcribes a polymorphic region of the DNA when extended.

5. The method as in claim 4, wherein the primer comprises a biotinylated primer and the transcribing step produces biotinylated DNA.

6. The method as in claim 3, further comprising binding the transcribed target nucleic acid sequence to a substrate.

7. The method as in claim 6, wheren the substrate comprises a streptavidin coated surface, support, plate or slide.

8. The method as in claim 6, further comprising removing unbound portions of target nucleic acid sequence.

9. The method as in claim 6, further comprising probing the bound target nucleic acid sequence using a sequence-tagged hybridization probe.

10. The method as in claim 9, wherein the target comprises DNA having at least one point mutation and the probing comprises binding the probe to said at least one point mutation of the DNA.

11. The method as in claim 9, wherein the target comprises wild type DNA and the probing comprises binding the probe to the wild type DNA.

12. The method as in claim 9, further comprising removing non-specifically bound probe.

13. The method as in claim 9, wherein each quantum dot has an attached oligonucleotide tag and labeling comprises binding each tag with a complementary sequence of each sequence-tagged hybridization probe.

14. The method as in claim 13, further comprising removing unbound quantum dots.

15. The method as in claim 13, wherein detecting comprises scanning the substrate with resolution capable of detecting fluorescence emitted by single quantum dots.

16. The method as in claim 15, further comprising quantitating the target nucleic acid sequence by analyzing the detected emitted fluorescence, wherein analyzing comprises counting the number of quantum dots within an area of scanned substrate.

17. A method of detecting the presence of at least one target nucleic acid sequence in a sample, said method comprising:
labeling at least one target nucleic acid sequence with at least one quantum dot;
detecting the labeled target nucleic acid sequence by detecting fluorescence emitted by the at least one quantum dot, wherein the detection of fluorescence in the sample indicates the presence of at least one target nucleic acid sequence; and
quantitating the target nucleic acid sequence by analyzing the detected emitted fluorescence.

18. A method of detecting the presence of a target nucleic acid sequence in a sample, said method comprising:
transcribing a target nucleic acid sequence using a primer that is complementary to a portion of said target nucleic acid sequence and that comprises an immobilizable label to form an immobilizable target nucleic acid sequence;
immobilizing said immobilizable target nucleic acid sequence on a solid support to form an immobilized target nucleic acid sequence;
probing said immobilized target nucleic acid sequence using a sequence-tagged hybridization probe, wherein said sequence-tagged hybridization probe is complementary to a portion of said target nucleic acid squence;
labeling said immobilized target sequence with a quantum dot conjugate, wherein said quantum dot conjugate comprises a quantum dot and a nucleic acid sequence that is complementary to a portion of said sequence-tagged hybridization probe; and
detecting the labeled immobilized target nucleic acid sequence by detecting fluorescence emitted by said quantum dot, wherein the detection of fluorescence in said sample indicates the presence of said target nucleic acid sequence.
